(19) United States
(12) Patent Application Publication
(10) Pub. No.: US 2019/0160035 A1
(43) Pub. Date: May 30, 2019

(54) METHOD OF TREATMENT

(71) Applicant: QBIOTICS LIMITED, Yungaburra (AU)

(72) Inventors: Peter Gordon PARSONS, Yungaburra (AU); Peter Frederick SCHMIDT, Yungaburra (AU); Charles Stewart LOWDEN, Yungaburra (AU); Justine Emma CAMPBELL, Yungaburra (AU); Victoria GORDON, Yungaburra (AU)

(21) Appl. No.: 16/320,762

(22) PCT Filed: Jul. 28, 2017
(86) PCT No.: PCT/AU2017/050791

§ 371 Date: Jan. 25, 2019

(30) Foreign Application Priority Data
Jul. 28, 2016 (AU) 2016902980

(51) Int. Cl. A61K 31/336; A61K 31/495; A61K 31/44; A61K 31/4439; A61K 31/5415; A61K 31/137; A61K 31/426; A61K 31/573; A61K 9/06; A61K 9/00; A61K 47/34; A61P 35/00

(52) U.S. Cl. CPC A61K 31/336; A61K 31/495; A61K 31/44; A61K 31/4439; A61K 31/5415; A61P 35/00; A61K 31/426; A61K 31/573; A61K 9/06; A61K 9/0019; A61K 47/34; A61K 31/137

(57) ABSTRACT

The present invention relates to methods of treating mast cell tumors and soft tissue sarcomas using 6,7-epoxy-4,5,9,12,13,20-hexahydroxy-1-tigliaen-3-one derivatives in combination with at least one other pharmaceutically active agent. In particular embodiments, the tigliaen-3-one derivatives are 12, 13-acyl or ether derivatives and the derivative compound is delivered in a localised manner to the tumor or sarcoma. In particular embodiments, the at least one other pharmaceutically active agent is selected from an antihistamine, an anti-inflammatory agent and mixtures thereof.

### FIELD OF THE INVENTION

The present invention relates to methods of treating mast cell tumors and soft tissue sarcomas using 6,7-epoxy-4,5,9,12,13,20-hexahydroxy-1-tigliaen-3-one derivatives in combination with at least one other pharmaceutically active agent. In particular embodiments, the tigliaen-3-one derivatives are 12,13-acyl or ether derivatives and the derivative compound is delivered in a localised manner to the tumor or sarcoma.

### BACKGROUND OF THE INVENTION

Despite significant effort being expended on the research and development of cancer drugs, there remains a significant need for new cancer therapies, particularly for solid tumors.

Surgery and ionising radiation are the current standards of care for local treatment of solid tumors. However, only one third of all cancer patients are successfully treated using these methods and side effects including scaring and disfigurement due to surgery, nausea, vomiting, damage to epithelial surfaces, infertility and disfigurement due to radiation can have significant detrimental effects. The effectiveness of surgery and radiation is also dependent on the condition of the patient, the proximity and/or infiltration of tumors into adjacent tissues, inaccessibility, large tumor volume and intolerance of normal tissue to repeated courses of treatment. There remains a need for better local therapies for a wide range of solid tumors.

Companion animals have also come to play an important role in the lives of many people and companion animal ownership is growing worldwide. Cancer in companion animals is a key concern for both veterinarians and pet owners. According to the Morris Animal Foundation, cancer is the leading cause of death for pets over the age of two. It has been estimated that, worldwide, as many as 1 in 4 dogs and 1 in 6 cats will develop cancer at some time in their lives and almost 50% of dogs over the age of 10 will die of the disease. Despite the willingness of pet owners to pursue and pay for cancer therapies, many veterinarians recognise cancer amongst the most difficult and least successful areas of therapy.

Mast cell tumors (MCT) are the most common form of cancer in dogs representing approximately 16 to 21% of all canine cutaneous cancers. This is followed by soft tissue sarcoma (STS) making up approximately 15%. The standard of care for MCT and STS is surgical removal of the tumor, usually with wide margins. When this is not possible, one or a combination of chemotherapy, radiotherapy and cytoreductive surgery is undertaken. These therapeutic approaches are often expensive, have a low success rate and result in moderate to severe adverse side effects. Consequently, there remains a need for efficacious therapy with improved quality of life outcomes.

### SUMMARY OF THE INVENTION

The present invention is predicated at least in part on the discovery that 6,7-epoxy-4,5,9,12,13,20-hexahydroxy-1-tigliaen-3-one derivatives in combination with at least one other therapeutic agent are particularly effective in treating MCT and STS by local administration.

In a first aspect, the present invention provides a method of treating a MCT or a STS in a subject comprising administering to the subject a compound of formula (I):

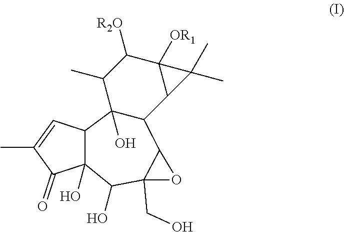

wherein R₁ and R₂ are independently selected from —C₁₋₂₀alkyl, —C₂₋₂₀alkenyl, —C₂₋₂₀ alkynyl, —C(O)C₁₋₂₀alkyl, —C(O)C₂₋₂₀alkenyl and —C(O)C₂₋₂₀alkynyl; wherein each alkyl, alkenyl and alkynyl may be optionally substituted with one or more groups selected from —OH, —SH, —OC₁₋₄alkyl, —SC₁₋₄alkyl, -halo, —CN, —CF₃, —CHF₂, —CH₂F, —OCF₃, —OCHF₂, —OCH₂F, —SCF₃, —SCHF₂, —SCH₂F, —CO₂H, —CO₂C₁₋₄alkyl, —NH₂, —NH(C₁₋₄alkyl), —N(C₁₋₄alkyl)₂ and —NO₂;

or a pharmaceutically acceptable salt thereof in combination with at least one other therapeutic agent.

In another aspect of the invention, there is provided a kit comprising a compound of formula (I) or a pharmaceutically acceptable salt thereof and at least one second pharmaceutically active agent.

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 25%, 20%, 15% or 10% to a reference quantity, level, value, dimension, size, or amount.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The term “alkyl” refers to optionally substituted linear or branched hydrocarbon groups having 1 to 20 carbon atoms. Where appropriate, the alkyl group may have a specified number of carbon atoms, for example, —C₁-C₆ alkyl which includes alkyl groups having 1, 2, 3, 4, 5 or 6 carbon atoms in linear or branched arrangements. Non-limiting examples of alkyl groups include methyl, ethyl, propyl, isopropyl, 1-methylpropyl, butyl, s- and t-butyl, pentyl, 2-methylbutyl, 3-methylbutyl, hexyl, 2-methylpentyl, 3-methylpentyl, 4-methylpentyl, 2-ethylbutyl, 3-ethylbutyl, heptyl, octyl, nonyl, decyl, undecyl, dodecyl, tridecyl, tetradecyl, pentadecyl, hexadecyl, heptadecyl, octadecyl, nonadecyl and eicosyl.

The term “alkenyl” refers to optionally substituted, unsaturated linear or branched hydrocarbons, having 2 to 20 carbon atoms and having at least one double bond. Where appropriate, the alkenyl group may have a specified number of carbon atoms, for example, C₂-C₆ alkenyl which includes alkenyl groups having 2, 3, 4, 5 or 6 carbon atoms in linear or branched arrangements. Non-limiting examples of alkenyl groups include, ethenyl, propenyl, isopropenyl, 1-methylprop-1-enyl, butenyl, 2-methylprop-1-enyl, 2-methyl-prop-2-enyl, pentenyl, hexenyl, hept-1,3-diene, hex-1,3-diene, non-1,3,5-triene and the like.

The term “alkynyl” refers to optionally substituted unsaturated linear or branched hydrocarbons, having 2 to 20 carbon atoms, having at least one triple bond. Where appropriate, the alkynyl group may have a specified number of carbon atoms, for example, C₂-C₆ alkynyl which includes alkynyl groups having 2, 3, 4, 5 or 6 carbon atoms in linear or branched arrangements. Non-limiting examples include ethynyl, propynyl, butynyl, pentynyl and hexynyl.

The term “acyl” as used herein includes a carbonyl group C(═O), substituted by an alkyl, alkenyl or alkynyl group. For example, an acyl group includes —C(O)C₁₋₂₀alkyl, —C(O)C₂₋₂₀alkenyl and —C(O)C₂₋₂₀alkynyl.

The term “in combination with” is used herein to refer to therapies that are used together where the active compounds are biologically active at the same time or at overlapping times with each other. The combination may be in a single composition or each component of the composition may be in a separate composition for simultaneous or sequential administration. In the combination, one component may be administered only once during the treatment period, while other components may be administered multiple times on days before, on or after the singly administered component.

The compounds of formula (I) may be in the form of pharmaceutically acceptable salts. It will be appreciated however that non-pharmaceutically acceptable salts also fall within the scope of the invention since these may be useful as intermediates in the preparation of pharmaceutically acceptable salts or may be useful during storage or transport. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that compounds of formula (I) possess asymmetric centres and are therefore capable of existing in more than one stereoisomeric form. The compounds may therefore be in substantially pure isomeric form at one or more asymmetric centres e.g., greater than about 90% ee, such as about 95% or 97% ee or greater than 99% ee, as well as mixtures, including racemic mixtures, thereof. Such isomers may be obtained by isolation from natural sources, by asymmetric synthesis, for example using chiral intermediates, or by chiral resolution. The compounds of the invention may exist as geometrical isomers. The invention also relates to compounds in substantially pure cis (Z) or trans (E) forms or mixtures thereof.

The compounds of formula (I) may be obtained by isolation from a plant or plant part, or by derivatisation of the isolated compound, or by derivatisation of a related compound as set out in WO 07/070985 and WO2014/169356.

### Methods of the Invention

The present invention relates to the treatment of MCT or STS in a subject. MCT, also known as mastocytoma, is a round cell tumor consisting of mast cells or is an accumulation or nodule of mast cells that resembles a tumor. MCTs may be malignant or benign. The grade of the tumor may be determined by biopsy:

Grade I—well differentiated and mature cells with a low potential for metastasis.

Grade II—intermediately differentiated cells with potential for local invasion and moderate metastatic behaviour.

Grade III—undifferentiated, immature cells with a high potential for metastasis.

The disease is also staged according to the WHO system:

Stage I—a single skin tumor with no spread to lymph nodes.

Stage II—a single skin tumor with spread to lymph nodes in the surrounding area.

Stage III—multiple skin tumors or a large tumor invading deep into the skin with or without lymph node involvement.

Stage IV—a tumor with metastasis to the spleen, liver, or bone marrow, or with the presence of mast cells in the blood.

In cats the MCT may be histiocytic, appearing as subcutaneous nodules.

The STS may be a fibrosarcoma, malignant fibrous hystiocytoma, dermatofibrosarcoma, liposarcoma, myxosarcoma, rhabdomyosarcoma, malignant mesenchymoma, leiomyosarcoma, hemangiosarcoma, Karposi's sarcoma, lymphangiosarcoma, undifferentiated sarcoma, synovial sarcoma, peripheral nerve sheath tumor, neurofibrosarcoma, pleomorphic sarcoma, extraskeletal chondrosarcoma, extraskeletal osteosarcoma, embryonyl sarcoma, infantile haemangiopericytoma (perivascular wall tumor), malignant schwannoma, neurogenic sarcoma, alveolar soft part sarcoma, extraskeletal myxoid chondrosarcoma or extraskeletal mesenchymal sarcoma, especially peripheral nerve sheath tumor, perivascular wall tumor, liposarcoma, myxosarcoma, pleomorphic sarcoma, malignant mesenchymoma and undifferentiated sarcoma. In some embodiments, the STS is not a fibrosarcoma, malignant fibrous histiocytoma or a dermatofibrosarcoma.

The MTC or STS may be located anywhere on or in the body of the subject. In some embodiments, the MCT or STS is located on the surface of the body or in a location accessible from the surfact of the body. In particular embodiments, the tumor or sarcoma is a cutaneous or subcutaneous tumor.

The compounds useful in the method of the invention are tigliaenone compounds of formula (I):

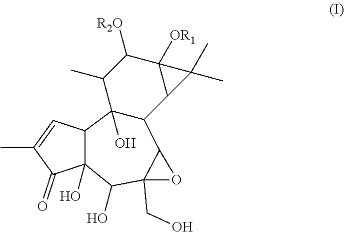

wherein R₁ and R₂ are independently selected from —C₁₋₂₀alkyl, —C₂₋₂₀alkenyl, —C₂₋₂₀alkynyl, —C(O)C₁₋₂₀alkyl, —C(O)C₂₋₂₀alkenyl and —C(O)C₂₋₂₀alkynyl; wherein each alkyl, alkenyl and alkynyl may be optionally substituted with one or more groups selected from —OH, —SH, —OC₁₋₄alkyl, —SC₁₋₄alkyl, -halo, —CN, —CF₃, —CHF₂, —CH₂F, —OCF₃, —OCHF₂, —OCH₂F, —SCF₃, —SCHF₂, —SCH₂F, —CO₂H, —CO₂C₁₋₄alkyl, —NH₂, —NH(C₁₋₄alkyl), —N(C₁₋₄alkyl)₂ and —NO₂;

or a pharmaceutically acceptable salt thereof.

In particular embodiments, R₁ and R₂ are independently selected from —C₁₋₁₀alkyl, —C₂₋₂₀alkenyl, —C₂₋₁₀alkynyl, —C(O)C₁₋₁₀alkyl, —C(O)C₂₋₁₀alkenyl and —C(O)C₂₋₁₀alkynyl, especially —C(O)C₁₋₁₀alkyl, —C(O)C₂₋₁₀alkenyl and —C(O)C₂₋₁₀alkynyl, more especially —C(O)C₁₋₁₀alkyl and —C(O)C₂₋₁₀alkenyl. In some embodiments, R₁ is selected from —C(O)C₁₋₁₀alkyl, especially —C(O)C₁₋₆alkyl. In some embodiments, R₂ is selected from —C(O)C₁₋₁₀alkyl and —C(O)C₂₋₁₀alkenyl, especially —C(O)C₁₋₆alkyl and —C(O)C₂₋₆alkenyl.

In particular embodiments, the compound of formula (I) is selected from:
12-tigloyl-13-(2-methylbutanoyl)-6,7-epoxy-4,5,9,12,13,20-hexahydroxy-1-tigliaen-3-one (Compound 1),
12,13-di-(2-methylbutanoyl)-6,7-epoxy-4,5,9,12,13,20-hexahydroxy-1-tigliaen-3-one (Compound 2),
12-hexanoyl-13-(2-methylbutanoyl)-6,7-epoxy-4,5,9,12,13,20-hexahydroxy-1-tigliaen-3-one (Compound 3), and
12,13-di-hexanoyl-6,7-epoxy-4,5,9,12,13,20-hexahydroxy-1-tigliaen-3-one (Compound 4).

The subject is one that has a MCT or a STS and is a mammal, reptile, bird or fish. In some embodiments the subject is a human subject. In other embodiments, the subject is a companion animal, farm animal, captive wild animal or laboratory animal. Suitable companion animals include dogs, cats, horses, rabbits, mice, rats, hamsters, guinea pigs and birds. Suitable farm animals include cows, pigs, sheep, deer, alpaca, goats, llama, chickens, geese, turkeys, ducks, quails, partridge and the like. Suitable captive wild animals include animals kept in zoos such as zebra, giraffe, antelope, buffalo, lions, tigers, cheetahs, leopards, hyena, dingo, monkeys, koalas, kangaroos, wombats, possums, Tasmanian devils, platypus, echidnas and the like.

In particular embodiments, the subject is a companion animal or a farm animal, especially a dog, cat, horse, cow or pig, more especially a dog, cat or horse, most especially a dog or cat.

Whilst the compound of formula (I) may be administered in any manner that allows treatment of the tumor, in particular embodiments, the administration is localised administration, for example, directly onto or into the tumor. In some embodiments, The localised administration is topical administration. In some embodiments, the localised administration is direct administration into the tumor. In particular embodiments, the compound of formula (I) is administered by injection directly into the tumor (intratumoral). The compound of formula (I) may be administered to one or more sites in the tumor. In this embodiment, the intratumoral administration may be to insert the syringe needle into a site in the tumor then move the needle radially while injecting the compound of formula (I) to ensure that the compound is dispersed throughout the tumor.

In other embodiments, the administration is a topical administration where the compound of formula (I) is administered topically at the site of the MCT or STS in the form of a gel, ointment, cream lotion, dressing, transdermal patch or poultice. In particular embodiments, the administration is topical administration of a gel, lotion, cream or ointment formulation at the site of the tumor.

The compound of formula (I) is administered in an effective amount. An “effective amount” means an amount necessary at least partly to attain the desired response, for example, to reduce the size of the tumor or to destroy the tumor in total. The amount varies depending upon the health and physical condition of the individual to be treated, the taxonomic group of individual to be treated, the formulation of the composition, the size of the tumor, the assessment of the medical situation, and other relevant factors. It is expected that the amount will fall in a relatively broad range that can be determined through routine trials. An effective amount, for example, may lie in the range of about 0.1 ng per kg of body weight to 1 g per kg of body weight per dosage. The dosage is preferably in the range of 1 μg to 0.5 g per kg of body weight per dosage, such as is in the range of 0.1 μg to 100 mg per kg of body weight per dosage. In one embodiment, where the dosage is administered intratumorally, the dosage is in the range of 0.1 mg to 5 mg per kg, especially 0.1 to 1 mg/kg dosage, such as 0.25 mg/kg dosage. In another embodiment, the dosage is in the range of 0.01 mg to 20 mg per dosage, especially 0.05 to 10 mg per dosage, more especially about 0.1 to about 5 mg per dosage. Dosage regimes may be adjusted to provide the optimum therapeutic response. For example, in some embodiments, where administration is intratumoral, the compound of formula (I) is administered once and the progress of treatment monitored. In some embodiments, if the tumor does not completely resolve or if the tumor recurs, a second dose may be administered. In some embodiments, where the administration is topical, the topical compound formulation may be administered directly onto the site of the tumor in the form of a gel, cream, ointment or lotion. The frequency of treatment will depend on the tumor, its size, the subject being treated and the like. In some embodiments, a topical formulation may be applied weekly until the tumor is resolved. In other embodiments, the treatment may be a single treatment and a second treatment only administered if the tumor is not completely resolved.

While the compound of formula (I) may be administered neat, in preferred embodiments, the compound is formulated in a composition that allows the compound to be effectively administered to the tumor. In some embodiments of the present invention, the compound is formulated for administration by injection, especially by intratumoral injection. In other embodiments, the compound is formulated for topical application in the form of a gel, cream, ointment or lotion. The compound may be formulated as an injectable liquid, a gel, a cream, a lotion, an ointment or a topical liquid and include pharmaceutically acceptable carriers, excipients and adjuvants as necessary. By “pharmaceutically acceptable” is meant a solid or liquid filler or diluent that may be safely used. Depending upon the particular route of administration, a variety of carriers, well known in the art may be used.

Liquid form preparations include solutions, suspensions, and emulsions, for example, water or water-propylene glycol solutions. Injectable liquid preparations can be formulated as solutions in aqueous 1,2-propanediol, dimethylsulfoxide (DMSO), aqueous solutions of gamma cyclodextrin or 2-hydroxypropyl-beta-cyclodextrin, saline solution or polyethylene glycol solution, with or without buffer. A preferred range of pH is 3.5-4.5. Suitable buffers buffer the preparation at pH 3.5-4.5 and include, but are not limited to, acetate buffer and citrate buffer.

The compounds of formula (I) may thus be formulated for administration by injection, for example bolus injection, and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

In some embodiments, the compound of formula (I) is formulated for topical administration. For topical administration to the epidermis or other organ, the compounds of formula (I) may be formulated as gels, ointments, emulsions, pastes, creams or lotions, or as a transdermal patch. Gels may be prepared using suitable thickening agents and adding them to aqueous/alcoholic compositions, such as aqueous isopropanol compositions, of compound. Suitable thickening or gelling agents are known in the art, such as the polyvinyl carboxy polymer, Carbomer 940. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or coloring agents.

In some embodiments, the compound of formula (I) is administered once. In other embodiments, the tumor or sarcoma is monitored and after a period of time, for example, if residual tumor cells are present, a second dose may be administered. In some embodiments, if the tumor recurs, a second dose is administered. The second dose may be administered, for example, 1 month to 1 year after the initial dose.

The compound of formula (I) is administered separately, either simultaneously or sequentially, or in the same composition as at least one other pharmaceutically active agent. In some embodiments, the at least one other pharmaceutically active agent is a supportive or prophylactic medication. The supportive medication may be administered prophylactically to reduce the likelihood of an adverse side effect or may be administered in response to a side effect or other medical event related to the treatment. For example, the compound of formula (I) may be administered in combination with one, two, three or more of an anti-inflammatory agent, an antihistamine, an analgesic, a hormone, an antiemetic, an anti-protozoal compound, an antifungal compound, a heart medication, an antibiotic and/or a proton pump inhibitor or other gastric-protecting medication. Suitable anti-inflammatory agents include non-steroidal anti-inflammatory drugs such as meloxicam, piroxicam, oxicam, aspirin, difunisal, ibuprofen, carprofen, dexibuprofen, naproxen, ketoprofen, indomethacin, tolmetin, mefenamic acid, numisulide and the like and corticosteroids such as hydrocortisone, prednisolone, methylprednisolone, prednisone, budesonide, betamethasone, triamcinolone and dexamethasone. Suitable antihistamines include cetirizine, pyrilamine, loratidine, chlorpheniramine, diphenhydramine, fexofenadine, hydroxyzine, pheniramine, dexchlorpheniramine, ranitidine, cimetidine, famotidine, lafutidine, nizatidine, promethazine and roxatidine. Suitable antibiotics include beta-lactam antibiotics such as penicillin, ampicillin, amoxycillin, flucloxacillin, dicloxacillin, methacillin, carbenicillin and norocillin; cephalosporins such as cephalexin, cefacetrile, cefadroxil, cefaloglycin, cefalonium, cefalordidine, cefatrizine, ceaclor, cefproxil, cefuzonam, cefmetozole, loracarbef, cefminox, cefdinir, cefpodoxime, and cefpirome; carbapenems such as imipenem, meropenem, ertapenem, daripenem, panipenem and biapenem; aminoglycosids such as gentamicin, streptomycin, neomycin, kanamycin, vancomycin, erythromycin and asithromycin; oxazolidinones such as linezolid and posizolid, lincosamides such as clindamycin, quinolines such as oxolinic acid, ciprofloxacin, enoxacin, ofloxacin, lomefloxacin, levofloxacin and difloxacin; and sulfonamides such as sulfamethoxazole, sulfoadiazine and sulfacetamide, other antibacterials such as chlorhexidine or mixtures such as amoxyclav (amoxycillin and clavulinic acid). Suitable analgesics and sedatives include aspirin, tramadol, methadone, buprenorphine, morphine, codeine, fentanyl, acetylpromazine, lignocaine and the like. Suitable proton pump inhibitors include omeprazole and other gastric-protecting medications include histamine H2 receptor antagonist such as famotidine. Suitable hormones include hormone replacements such as stilboestrol. Suitable antiemetics include maropitant citrate. Suitable antiprotozoal compounds include metronidazole. Suitable antifungal compounds include miconazole or miconazole with chlorhexidine. Suitable heart medications include sotalol and propranolol.

In particular embodiments, especially where the tumor is a MCT, the compound of formula (I) may be administered in combination with an antihistamine. This is because, MCT may release histamine during treatment. In particular embodiments, the antihistamine is selected from cetirizine, loratidine, chlorpheniramine, dexchlorpheniramine, famotidine and ranitidine. The administration of the antihistamine may be by oral administration or by injection, for example, subcutaneous or intramuscular injection. Amounts of antihistamines include those commonly used in the art. For example, cetirizine may be administered at an amount of about 0.25 mg/kg and chlorpheniramine in an amount of around 5 mg/10 kg. The antihistamine may be administered at the time of administration of the compound of formula (I) and may be continued on a daily basis for 1 to 10 days or 1 to 8 days, or 1 to 5 days as determined by the physician or veterinarian.

In some embodiments, the compound of formula (I) is administered in combination with an anti-inflammatory drug. In particular embodiments, the compound of formula (I) may be administered in combination with an anti-inflammatory drug such as meloxicam, piroxicam, oxicam, aspirin, difunisal, ibuprofen, carprofen, dexibuprofen, naproxen, ketoprofen, indomethacin, tolmetin, mefenamic acid, numisulide or a corticosteroid such as hydrocortisone, prednisolone, methylprednisolone, prednisone, budesonide, betamethasone, triamcinalone and dexamethasone. In a particular embodiment, the compound of formula (I) is administered in combination with a corticosteroid, especially prednisolone. The corticosteroid may be administered in amounts commonly used and by normal administration routes. For example, oral administration of 0.5 mg/kg prednisolone twice a day for three days and then 0.5 mg/kg once a day for 3 to 5 days may be a suitable dosage regimen to start the day before, together with or following administration of compound 1. The dosage regimen may be determined by a physician or veterinarian.

Other medications such as famotidine and omeprazole may be used to reduce gastrointestinal side effects.

In some embodiments, the combination includes one compound of formula (I) together with one supporting medication.

In other embodiments, the combination includes one compound of formula (I) together with two supporting medications.

In other embodiments, the combination includes one compound of formula (I) together with three supporting medications.

In other embodiments, the combination includes one compound of formula (I) together with four supporting medications.

In other embodiments, the combination includes one compound of formula (I) together with five supporting medications.

In other embodiments, the combination includes one compound of formula (I) together with six supporting medications.

The at least one other medication may be divided into pre-treatment (Pre-Tx) and post-treatment with a compound of formula (I) (Post-Tx).

In some embodiments, where the tumor treated is a MCT, the subject may be treated with a pre-treatment regimen that includes an antihistamine, especially an antihistamine and an anti-inflammatory drug.

In some embodiments, where the tumor treated is a MCT, the subject may be treated with a post-treatment regimen that includes an antihistamine, especially an antihistamine and an anti-inflammatory drug.

In some embodiments, the compound of formula (I) is administered in combination with an antihistamine, wherein the antihistamine is administered on the day of treatment with the compound of formula (I), prior to treatment and/or subsequently after treatment and/or on each day post-treatment for 1 to 10 days, 1 to 8 days, 1 to 6 days or 1 to 5 days. Optionally, the antihistamine compound may be administered on the day preceding treatment with the compound of formula (I).

In some embodiments, where the antihistamine is administered, the antihistamine may be an H1 antihistamine. In some embodiments, the antihistamine given before treatment with formula (I) one treatment day (Day 0) is different from the antihistamine given as a pre-treatment on Day −1 and as a post-treatment on day of Days 0 to 5. In particular embodiments, the pre-treatment (Day −1) and post-treatment doses of antihistamine are administered orally at a dosage normally provided for that antihistamine, for example, 0.25 mg/kg oral cetirizine. In some embodiments, a higher dosage of antihistamine is provided on treatment day prior to administration of the compound of formula (I). The higher dosage may be administered by any suitable means, such as oral or parenteral. In some embodiments, the higher dosage of antihistamine is delivered intramuscularly. The higher dosage antihistamine may be the same or different to the pre-treatment/post-treatment antihistamine. In one example, the pre-treatment antihistamine administered on Day 0 is an intramuscular dose of 5 mg for subjects ≤10 kg or 5 mg/10 kg of chlorpheniramine.

In some embodiments, in addition to antihistamine, the combination therapy further comprises an anti-inflammatory and/or analgesic compound.

In some embodiments, the anti-inflammatory compound is administered prior to treatment with the compound of formula (I) on the day of treatment, and then on subsequent days for from 1 to 5 days. Optionally, the anti-inflammatory compound is administered on the day prior to treatment with the compound of formula (I).

In some embodiments, the anti-inflammatory compound is a non-steroidal anti-inflammatory drug that optionally has some analgesic properties, such as meloxicam. In others embodiments, the anti-inflammatory drug is a corticosteroid such as prednisolone. The dosage of anti-inflammatory is conveniently administered by oral delivery, particularly if administered on Day 1 prior to treatment or days 1 to 5 post-treatment. A normal dosage of anti-inflammatory drug may be administered, for example, 0.1 mg/kg body weight twice per day for Days −1 to Day 3 post-treatment, then once per day Days 4 to 6 post-treatment.

On the day of treatment with a compound of formula (I), the anti-inflammatory dosage may be maintained the same as pre-treatment or post-treatment dosages. Alternatively, a higher dosage of anti-inflammatory drug may be administered on the day of treatment with a compound of formula (I) as a pre-treatment. For example, a pre-treatment on Day 0 of 0.2 mg/kg body weight meloxicam delivered subcutaneously.

In some embodiments, the combination therapy may include a compound that has gastric protection properties, for example, a proton pump inhibitor such as omeprazole or a histamine H2 receptor antagonist such as famotidine. Such a treatment may be given for up to 10 days post-treatment with compound of formula (I), especially 8 days post-treatment, 6 days post-treatment or 5 days post-treatment. Optionally, the compound having gastric protective properties may be administered as a pre-treatment on the day before treatment with a compound of formula (I) and as a pre-treatment and/or post-treatment on the day of treatment. Suitable dosages include 1.0 mg·kg bwt oral omeprazole once a day or 0.5 mg/kg bwt oral famotidine twice per day.

Further treatments may be administered on the day of treatment with the compound of formula (I), for example, analgesics and/or sedatives and/or antiemetics.

In some embodiments, the tumor is a soft tissue sarcoma and the at least one other pharmaceutically active agent is an non-steroidal anti-inflammatory drug such as methoxicam or carbofem.

In some embodiments, the anti-inflammatory drug is provided pre-treatment with the compound of formula (I) and/or post-treatment with the compound of formula (I). The anti-inflammatory drugs may also be administered in oral form, especially for post-treatment administration. However, pre-treatment administration on the day of treatment may be delivered parenterally, for example, by subcutaneous injection. Examples of suitable anti-inflammatory drugs include meloxicam and carprofen.

In some embodiments, the anti-inflammatory drug is administered together with an analgesic. Suitable analgesics include opioid analgesics such as tramadol.

In another aspect, there is provided a use of a compound of formula (I):

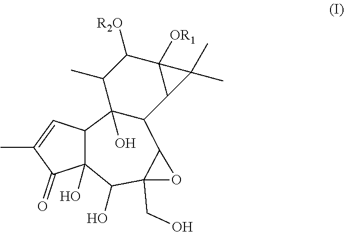

wherein R₁ and R₂ are independently selected from —C₁₋₂₀alkyl, —C₂₋₂₀alkenyl, —C₂₋₂₀alkynyl, —C(O)C₁₋₂₀alkyl, —C(O)C₂₋₂₀alkenyl and —C(O)C₂₋₂₀alkynyl; wherein each alkyl, alkenyl and alkynyl may be optionally substituted with one or more groups selected from —OH, —SH, —OC₁₋₄alkyl, —SC₁₋₄alkyl, -halo, —CN, —CF₃, —CHF₂, —CH₂F, —OCF₃, —OCHF₂, —OCH₂F, —SCF₃, —SCHF₂, —SCH₂F, —CO₂H, —CO₂C₁₋₄alkyl, —NH₂, —NH(C₁₋₄alkyl), —N(C₁₋₄alkyl)₂ and —NO₂;

or a pharmaceutically acceptable salt thereof; in the manufacture of a medicament for treating a MCT or STS in combination with at least one other pharmaceutically active agent.

In a further aspect of the present invention there is provided a compound of formula

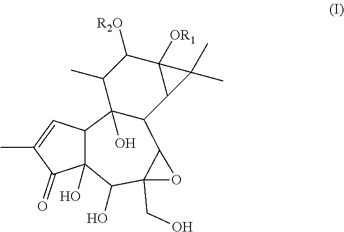

wherein R₁ and R₂ are independently selected from —C₁₋₂₀alkyl, —C₂₋₂₀alkenyl, —C₂₋₂₀alkynyl, —C(O)C₁₋₂₀alkyl, —C(O)C₂₋₂₀alkenyl and —C(O)C₂₋₂₀alkynyl; wherein each alkyl, alkenyl and alkynyl may be optionally substituted with one or more groups selected from —OH, —SH, —OC₁₋₄alkyl, —SC₁₋₄alkyl, -halo, —CN, —CF₃, —CHF₂, —CH₂F, —OCF₃, —OCHF₂, —OCH₂F, —SCF₃, —SCHF₂, —SCH₂F, —CO₂H, —CO₂C₁₋₄alkyl, —NH₂, —NH(C₁₋₄alkyl), —N(C₁₋₄alkyl)₂ and —NO₂;

or a pharmaceutically acceptable salt thereof; for use in treating a MCT or STS.

In another aspect of the present invention there is provided a kit comprising one or more dosages of a compound of formula (I) and at least one other pharmaceutically active agent. In some embodiments, the at least one other pharmaceutically active agent is an antihistamine and/or a corticosteroid. In some embodiments, the kit comprises one or more dosages of the at least one other pharmaceutically active agent. In a particular embodiment, the kit comprises one dose of a compound of formula (I) and 3 to 10 doses of an antihistamine and/or 3 to 10 doses of a corticosteroid.

In some embodiments, the one or more dosages of the compound of formula (I) is in the form of an injectable composition. In other embodiments, the compound of formula (I) is in the form of a topical composition, such as a gel, ointment, cream or lotion.

The invention will now be described with reference to the following Examples which illustrate some preferred aspects of the present invention. However, it is to be understood that the particularity of the following description of the invention is not to supersede the generality of the preceding description of the invention.

### EXAMPLES

### Example 1: Treatment of Dogs with Round Cell Tumors at a Dosage of 0.4 mL/cm³

### Animal Population

Study dogs were managed similarly and with due regard for their welfare. Animals were enrolled in the study in accordance with instructions provided in Australian Pesticides and Veterinary Medicines Authority (APVMA) small-scale trials permit PER7250 (unregistered drug use). All animals were client-owned, treated by Australian registered veterinarians, and cases managed under their direct supervision. An enrolled dog could be removed from the study prior to study completion under the following circumstances:
1. If at any time during the study, in the opinion of the attending veterinarian, the dog's welfare is at risk due to non-response of the tumor to Compound 1 treatment or due to a serious adverse event, the veterinarian could elect to withdraw the dog from the study. Following withdrawal, the dog's attending veterinarian could then administer supportive treatment and veterinary care that they deem is appropriate and in the best interest of the animal.
2. The owner elected to voluntarily withdraw the dog from the study.

Dogs were selected for the study if they met the criteria in Table 1 below.

TABLE 1
Study inclusion criteria

The animal was of any breed and either gender, but females were non-
pregnant, non-lactating, and not intended for breeding during the study
(4 weeks).
The dog was ≥1 year of age at the Day 0 visit.
The dog was expected to be manageable and cooperative with study
procedures.
The dog was generally healthy with no clinically significant abnormalities
that would interfere with study objectives, as determined by the pre-
enrolment history, and physical examinations at the screening visit (e.g.
debilitating gastrointestinal disease, coagulopathy, etc.)
The dog had a life expectancy of at least 3 months.
The dog had at least one skin RCT. In the case of multiple tumors, the
attending veterinarian selected one tumor to become the focus of this
study.
The surface of the selected RCT was intact with the expectation of
minimal drug leakage from the tumor surface upon injection
The selected tumor was characterised and recorded as a cutaneous
MCT or subcutaneous MCT, histiocytoma, plasmacytoma or lymphoma
based on palpation and fine needle aspirate (FNA) for that tumor.
Confirmation through laboratory FNA was permissible as required.
The selected RCT was not at the site of a previously resected tumor.
The dog must not have received systemic chemotherapy or radiotherapy
as treatment for a malignancy within 2 months preceding the planned
treatment day.
The dog must not have received any immunosuppressants (e.g.
corticosteroids, cyclosporine, etc.) within the 14 days preceding
the planned treatment day.
The dog must not have received prior treatment of the target tumor
with Compound 1, or received other Compound 1 for the treatment of
other tumors within 2 months preceding the planned treatment day.
The dog had no known sensitivity to other medications used in the
study
The total calculated volume of the selected skin RCT was expected
to be <12.5 cm³ at the Day 0 visit, (confirmed before drug injection on
that day)
The calculated dose of drug for the dog was ≤0.25 mg drug per kg at
the Day 0 visit (maximum dose of 5 mg drug)

### Exclusion Criteria:

Dogs that were debilitated, suffering from disease other than RCT or injury, fractious or otherwise unsuitable for inclusion in the study, in the opinion of the attending veterinarian, were not enrolled. Animal enrolment details are provided in Table 2.

Removal of Animals from Assessment

No animals were removed from the study.

### Animal Enrolment Details:

TABLE 2
Animal Enrolment Details

| | |
|---|---|
| Species | Canis lupus familiaris (dog) |
| Number | 12† |
| Weight | 5.4-37.9 kg (median 22.7 kg) |
| Sex | 9 females (8 neutered) and 3 males (1 neutered) |
| Age | 4-12 yrs (median 9.0 yrs) (1 unknown) |
| Source | Client owned |
| Identification method | Enrolment no. |

†8 cutaneous MCT and 4 subcutaneous MCT

### Drug Treatment

Compound 1 was prepared for intratumoral injection by preparation of a composition comprising 1 mg/mL Compound 1 in a 40% propylene glycol solution with 30 mM acetate buffer, pH 4.2. The dosage level was 0.4 mL/cm³ of tumor.

### Dose Calculation:

The drug was administered to each dog as 0.4 mL per cm³ of tumor volume (40% v/v tumor) as determined on the day of dosing (Day 0). Tumor volume was determined using the modified ellipsoidal calculation=½×length×width×depth (cm) (Monga et al, 2000; Celikoglu et al, 2008).

### Dose Preparation:

The required volume of the designated dose concentration of the drug was drawn into an appropriately sized and graduated sterile syringe.

### Method of Drug Administration:

Study animals were dosed according to the treatment regime detailed in Table 4.

TABLE 3
Treatment regime

| | Dose Volume | | | No. of |
|---|---|---|---|---|
| Drug Details | (mL/cm³ of tumor) | Route | Frequency | animals |
| Compound 1 | 0.4 | Intratumoral | once | 12 |
| injection | | | | |
| 1.0 mg/mL | | | | |

Wherever possible, the tumor and a 2 cm border surrounding the tumor was shaved prior to treatment. To deliver the drug, the needle of the dosing syringe was inserted into the tumor at the injection point and moved in a radial manner in 2- and 3-dimensions. Treatment administration was recorded on the individual “Case Report Form”. Study dogs were clinically examined at 2-4 and 24 hours, and 7 (±2), 14 (±2) and 28 (±2) days post-treatment (and unscheduled visits at attending veterinarian discretion).

### Prophylactic and Supportive Medications

MCT can give rise to paraneoplastic disease associated with the release of bioactive substances from mast cell granules (degranulation) (Fox et al, 1990; Ishiguro et al, 2003). These substances include histamine, heparin and proteolytic enzymes. In addition, a number of proinflammatory and tissue destructive enzymes may be released (Pimental et al, 2011). These bioactive substances can cause oedema, ulceration, and swelling at the tumor site, and possibly delayed wound healing and local coagulation abnormalities (Blackwood et al, 2012). In rare cases, massive release of histamine from neoplastic mast cells can result in an acute anaphylactoid reaction and episodic collapse (Blackwood et al, 2012).

Each supervising veterinarian was given latitude to provide and record prophylactic and supportive medications as deemed appropriate for managing potential paraneoplastic events and tumor site necrosis following treatment with Compound 1. The medications included H1 and H2 antihistamines (cetirizine, loratidine, chlorpheniramine, dexchlorpheniramine, ranitidine), proton pump inhibitors (omeprazole), non-steroidal anti-inflammatory drugs (NSAIDs) (meloxicam, carprofen), opioid analgesics (tramadol, methadone, buprenorphine), antibiotics (amoxicillin/clavulinic acid) and corticosteroids (prednisolone).

The schedule of support medications is shown in Table 4.

TABLE 4
Prophylactic and Supportive Medications

| | | | | Dog ID | | | | | | | | | | | |
|---|---|---|---|---|---|---|---|---|---|---|---|---|---|---|---|
| Prophylactic and Supportive Medications | | | | C03- | C03- | C03- | C03- | C03- | C03- | C03- | C03- | C03- | C03- | C03- | C03- |
| | Active ingredient | Tradename | Delivery | N-01 | N-02 | N-03 | T-01 | T-02 | T-03 | T-04 | T-05 | T-06 | T-07 | T-08 | T-09 |
| Pre- | Cetirizine | ZYRTEC | PO | | | | X | | X | | | | | | |
| Tx | Loratidine | CLARATYNE | PO | | | | | | | | | | | | X |
| | Chlorpheniramine | IRAMINE | PO | X | X | X | | | | | | | | | |
| | Chlorpheniramine | HISTAMIL | IM | | | | X | X | X | X | X | X | X | X | X |
| | Chlorpheniramine | NIRAMINE | IM | X | X | X | | | | | | | | | |
| | Ranitidine | ZANTAC | PO | | | | | | | | | | | X | |
| | Omeprazole | LOSEC | PO | X | X | X | | | | | | | | | |
| | Methadone | METHONE | SC | X | | X | | | | | | | | | |
| | Meloxicam | METACAM LA | SC | | | | X | X | X | X | X | X | X | | X |
| | Meloxicam | METACAM SA | SC | X | X | X | | | | | | | | X | |
| Post- | Cetirizine | ZYRTEC | PO | | | | X | | X | | | | | | |
| Tx | Loratidine | CLARATYNE | PO | | | | | | | | | | | | X |
| | Chlorpheniramine | IRAMINE | PO | X | X | X | | X | | X | X | X | X | X | |
| | Dexchlorpheniramine | POLARAMINE | PO | | | | | | | | | | | X | |
| | Ranitidine | ZANTAC | PO | | | | | | | | | | | X | |
| | Omeprazole | LOSEC | PO | X | X | X | | | | | | | | | |
| | Meloxicam | METACAM | PO | X | | | | | | | X | X | | | |
| | Carprofen | RIMADYL | PO | | | | X | X | X | X | | | X | X | X |
| | Tramadol | TRAMADOL | PO | | | | X | X | X | | | X | X | X | X |
| | Buprenorphine | TEMGESIC | SC | X | X | | | | | | | | | | |
| | Amoxycillin/Clavulanic | AMOXYCLAV | PO | | | | | | | | | | | X | |
| | Acid | | | | | | | | | | | | | | |
| | Prednisolone | PREDNIL† | PO | | | X | | | | | | | | | |
| | Prednisolone | MACROLONE† | PO | | | | | X | | | | | | | |
| | Metronidazole | METROGYL¥ | PO | | | | | X | | | | | | | |

†for pre-existing skin condition;
¥for diarrhoea (possibly stress or Giardia sp. related);
ID—identification;
Pre-Tx = pre-treatment;
Post-Tx = post-treatment;
PO = per os (oral);
IM = intramuscular;
SC = subcutaneous

### Study Schedule

This schedule in Table 5 approximates the conduct of activities for each enrolled dog. All scheduled and unscheduled events during the study were recorded.

TABLE 5
Summary of study activities

| Approx. | |
|---|---|
| Study | |
| Day | Activities |
| Screening | A dog was enrolled if it met inclusion criteria (including |
| visit | confirmed diagnosis of an RCT via fine needle aspirate |
| | (FNA). A clinical examination and tumor assessment |
| | was performed. |
| Pre- | Owners' consent was obtained for dogs presenting to the |
| treatment | clinic that were likely to be suitable for enrolment in the |
| | study (before treatment). Prophylactic medications as |
| | deemed appropriate by the supervising veterinarian |
| | were provided. |
| Day 0 | Treatment was administered in accordance with the Day 0 |
| | tumor volume measurement. Supportive medications as |
| | deemed appropriate by the supervising veterinarian were |
| | prescribed. Dogs were either hospitalised overnight or |
| | discharged from hospital into client care (if condition |
| | was stable). |
| Day 1 | A clinical examination was performed. |
| Day 7 ± 2 | A clinical examination was performed, a tumor assessment |
| | recorded. |
| Day 14 ± 2 | A clinical examination was performed, a tumor assessment |
| | recorded. |
| Day 28 ± 2 | A clinical examination was performed, a tumor assessment |
| | recorded. |

### Efficacy Evaluation

Determination of efficacy was based on objective tumor measurements made according to the Response Evaluation Criteria in Solid Tumors (RECIST) v.1.1 guideline (Eisenhauer et al, 2009) using the longest unidirectional tumor measurement (diameter). The RECIST v.1.1 guideline states use for a minimum baseline measure of 10 mm. This was not a stipulation for dog enrolment in this study as long as the tumor was sufficiently delineated for measurement. Measurements were recorded using digital calipers at pre-treatment and at 7(±2), 14(±2) and 28(±2) days post treatment. The length (cm) was designated the longest measurable diameter; the width (cm) was the tumor at the widest point perpendicular to the length; and gentle manipulation of the MCT to measure the depth (cm) using the calipers was considered acceptable. On occasion, additional data was collected during an unscheduled visit. Response to therapy was defined as complete response (CR, resolution of the target lesion), partial response (PR, at least 30% decrease in the longest diameter of target lesion), stable disease (SD, decrease in the target lesion of less than 30% or increase of the target lesion less than 20%) or progressive disease (PD, greater than 20% increase in the target lesion).

### Safety Evaluation:

Clinical parameters such as bodyweight (kg), rectal temperature (° C.), heart rate (HR) per minute, respiratory rate (RR) per minute, general demeanor and body function (e.g. eating, drinking, urine and stools), were collected on each enrolled dog at screening, immediately pre-treatment, at approximately 2-4 and 24 hours post-treatment, and at 7(±2), 14(±2) and 28(±2) days post treatment. An adverse event (AE) was defined as any unfavorable and unintended sign, symptom, or disease temporally associated with the use of the drug, whether or not related to the product (VCOG-CTCAE, 2011). AE were graded according to the VCOG-CTCAE classification which is a descriptive severity scale (grade system) used for AE reporting in dogs treated with antineoplastic therapy.

### Efficacy Evaluation

Patient demographics for the twelve (12) evaluable cases are presented in Table 6. Only eleven (11) animals were available for efficacy assessment. One (1) dog (C03-T-07) died during the course of study conduct.

TABLE 6
Patient demographics

| | | | BW (kg) | | | |
|---|---|---|---|---|---|---|
| | | | Immediate | | | |
| | | Age | Pre- | | | |
| Dog ID | Breed | (yrs) | treatment | Sex | Tumor Type | Tumor location |
| C03-N-01 | Terrier X Pug | 12 | 37.9 | ME | Subcutaneous MCT | Left forelimb (radio- |
| | | | | | | ulnar) |
| C03-N-02 | Koolie | 10 | 22.8 | FN | Subcutaneous MCT | Right ventral flank |
| C03-N-03 | Staffordshire Terrier | 9 | 22.5 | ME | Cutaneous MCT | Midline ventral chest |
| C03-T-01 | Staffordshire Terrier | 4 | 25.0 | FE | Cutaneous MCT | Right side flank |
| C03-T-02 | Staffordshire Terrier X | 9 | 18.6 | MN | Cutaneous MCT | Left foot between toes |
| | | | | | | (3/4) |
| C03-T-03 | Boxer | 6 | 29.4 | FN | Cutaneous MCT | Right ventral chest |
| C03-T-04 | Staffordshire Terrier | 7 | 20.2 | FN | Cutaneous MCT | Right flank |
| C03-T-05 | Staffordshire Terrier | 10 | 18.2 | FN | Cutaneous MCT | Left flank |
| C03-T-06 | Mastiff X | 12 | 34.0 | FN | Cutaneous MCT | Left vulva |
| C03-T-07 | Staffordshire Terrier | unknown | 19.3 | FN | Subcutaneous MCT | Left caudal gluteal |
| C03-T-08 | Jack Russell Terrier X | 14 | 5.4 | FN | Subcutaneous MCT | Left ventral abdomen |
| C03-T-09 | Weimaraner | 9 | 32.2 | FN | Cutaneous MCT | Left side rump |

ID = identification;
X = cross;
BW = bodyweight;
FN = female neutered;
FE = female entire;
MN = male neutered;
ME = male entire;
MCT = mast cell tumor

Median age upon study enrolment was 9.0 years (range 4-12) and median bodyweight was 22.7 kg (range 5.4-37.9). Nine (9) females and three (3) males were enrolled. Nine (9) of these dogs were neutered. The most common breed represented were Staffordshire Terrier (or cross) (n=6). The recruitment method was for all eligible skin and subcutis RCT cases to be enrolled as presented at the participating clinics. Only cases of cutaneous and subcutaneous MCT were presented, eligible, and thus enrolled in this study. Calculated tumor volumes (cm³) and treatment details are summarised in Table 7.

TABLE 7
Calculated tumor volumes and treatment details

| | | Calculated | | | Dose |
|---|---|---|---|---|---|
| | | tumor volume | Date of | EBC46 conc. | Volume |
| Dog ID | Breed | (cm³) Day 0 | treatment | (mg/mL) | (mL) |
| C03-N-01 | Terrier X Pug | 2.35 | 01-Dec-14 | 1.0 | 0.94 |
| C03-N-02 | Koolie | 5.63 | 15-Dec-14 | 1.0 | 2.25 |
| C03-N-03 | Staffordshire Terrier | 0.05 | 10-Feb-15 | 1.0 | 0.02 |
| C03-T-01 | Staffordshire Terrier | 0.06 | 24-Nov-14 | 1.0 | 0.02 |
| C03-T-02 | Staffordshire Terrier X | 0.68 | 17-Dec-14 | 1.0 | 0.30 |
| C03-T-03 | Boxer | 0.16 | 28-Jan-15 | 1.0 | 0.10 |
| C03-T-04 | Staffordshire Terrier | 0.37 | 04-Feb-15 | 1.0 | 0.12 |
| C03-T-05 | Staffordshire Terrier | 0.10 | 12-Feb-15 | 1.0 | 0.10 |
| C03-T-06 | Mastiff X | 0.26 | 18-Feb-15 | 1.0 | 0.10 |
| C03-T-07 | Staffordshire Terrier | 2.53 | 19-Feb-15 | 1.0 | 1.00 |
| C03-T-08 | Jack Russell Terrier X | 1.07 | 26-Feb-15 | 1.0 | 0.42 |
| C03-T-09 | Weimaraner | 0.66 | 17-Mar-15 | 1.0 | 0.26 |

ID = identification;
X = cross;
conc. = concentration

The median tumor volume on Day 0 (the day of treatment) was 0.3 cm³ (range 0.05-5.63). At a dose level of 0.4 mL drug per cm³ tumor (40% v/v), the median dose volume was 0.1 mL (range 0.02-2.25).

RECIST v.1.1 results are summarised in Table 8. The raw data for RECIST assessment were collected on Day 0 (immediately pre-treatment) and on Day 28. Of the twelve (12) dogs treated, eleven (11) dogs were available for Day 28 RECIST classification. Six (6) dogs experienced complete response (CR), 3 dogs experienced partial response (PR), and 2 dogs experienced stable disease (SD). Dog C03-T-07 was not available for RECIST classification as it died on Day 4 post treatment. However, personal correspondence with the attending veterinarian indicated that no tumor cytology was identified in the autopsied injection site of this dog (Day 4).

TABLE 8
Response to therapy

| | | Calculated | | | | |
|---|---|---|---|---|---|---|
| | | tumor | Drug | | Day of | |
| | | volume (cm³) | conc. | Dose Volume | RECIST | RECIST |
| Dog ID | Breed | Day 0 | (mg/mL) | (mL) | assessment | classification |
| C03-N-01 | Terrier X Pug | 2.35 | 1.0 | 0.94 | 28 | CR |
| C03-N-02 | Koolie | 5.63 | 1.0 | 2.25 | 28 | SD |
| C03-N-03 | Staffordshire Terrier | 0.05 | 1.0 | 0.02 | 28 | PR |
| C03-T-01 | Staffordshire Terrier | 0.06 | 1.0 | 0.02 | 28 | SD |
| C03-T-02 | Staffordshire Terrier X | 0.68 | 1.0 | 0.30 | 28 | CR |
| C03-T-03 | Boxer | 0.16 | 1.0 | 0.10 | 28 | PR |
| C03-T-04 | Staffordshire Terrier | 0.37 | 1.0 | 0.12 | 28 | PR |
| C03-T-05 | Staffordshire Terrier | 0.10 | 1.0 | 0.10 | 28 | CR |
| C03-T-06 | Mastiff X | 0.26 | 1.0 | 0.10 | 28 | CR |
| C03-T-07† | Staffordshire Terrier | 2.53 | 1.0 | 1.00 | — | — |
| C03-T-08 | Jack Russell Terrier X | 1.07 | 1.0 | 0.42 | 28 | CR |
| C03-T-09 | Weimaraner | 0.66 | 1.0 | 0.26 | 28 | CR |

ID = identification;
conc. = concentration.
†Not available for RECIST classification. Dog died on day 4

### Safety Evaluation

With the exception of swelling and pain, local tissue pathology as a result of treatment (i.e. tumor necrosis) was not considered an adverse event (AE). AEs such as rapid breathing, mild tachycardia and post treatment lethargy are generally considered to be the result of the powerful local inflammatory response and systemic activation of the dog's immune response during the treatment response phase. In this study, 44 AEs were identified in 15 event types. The majority of identified AEs (28 out of 44; 61.3%) were represented by the ‘mild’ VCOG-CTCAE classification Grade 1 with no intervention required. With the exception of dog C03-T-07, a level of minimal medical intervention was required in only 3 cases. Dog C03-T-03 had a possible stress-related or Giardia-related occurrence of diarrhoea which subsided in the presence of metronidazole; dog C03-T-04 was instructed in a dietary modification for pre-existing intermittent vomiting; and dog C03-T-08 was prescribed a prophylactic antibiotic course (amoxicillin/clavulanic acid) during the post-treatment monitoring period. With the exception of dog C03-T-07, all reported AEs were transient in nature—resolved by 24-48 h post treatment in the majority of cases.

Dog C03-T-07 died during the course of the study. C03-T-07 was a female Staffordshire bull terrier. She had a moderate sized subcutaneous MCT on her left hind leg. She was treated with premedications (antihistamines and NSAIDs) prior to her drug treatment on 19th February 2015. The veterinarian reports that the following factors contributed to her death.
1. Treatment with acepromazine that would have contributed to hypotension and masked possible deterioration.
2. Delay in supportive medication due to sedation masking deterioration.
3. Inadequate antihistamine dose or inability/difficulty for owner to medicate.
4. Storm stress.

The attending veterinarian also stated that she did not believe that Compound 1 directly contributed to the death of dog C03-T-07.

### Example 2: Dosage of Compound 1

A multi-site, unmasked, uncontrolled, non-randomized dosage characterization study in dogs was performed under field conditions using dose de-escalation.

TABLE 9
Inclusion Criteria

Client owned dogs
Fine Needle Aspirate (FNA) confirmed cutaneous Mast Cell Tumor
(MCT)
MCT of stage 0, I, IIa by the World Health Organization staging system
for mast cell tumors (see Table 9).
Tumor at between 0.1 cm³ to 6.0 cm³
No subcutaneous mast cell tumors (Thomson et al.)
Dog life expectancy > 12 weeks
Weight of dog ≥ 5 kg
First presentation of MCT in the dog, or MCT that appears at a site
distinct to a previous MCT that was treated by surgical resection >6
months previously
Adequate hepatic, renal and haematological functions as determined
from clinical assessment.
No pregnant or lactating dogs, or dogs intended for breeding purposes
No dogs with evidence of serious systemic MCT disease
No administration of corticosteroids within 14 days prior to enrolment
in the study
No evidence of gastrointestinal bleeding or evidence of coagulopathy
No prior radiation therapy regimen or systemic chemotherapy regime for
treatment of the MCT
Informed owner consent

TABLE 10
WHO Clinical Staging System for Mast Cell Tumors

| Stage | Description | |
|---|---|---|
| 0 | One tumor incompletely excised from | a. without systemic signs |
| | the dermis without regional lymph | b. with systemic signs |
| | node involvement | |
| I | One tumor confined to dermis | a. without systemic signs |
| | without regional lymph node | b. with systemic signs |
| | involvement | |
| II | One tumor with regional lymph node | a. without systemic signs |
| | involvement | b. with systemic signs |
| III | Multiple dermal tumors or large | a. without systemic signs |
| | infiltrating tumor with or without | b. with systemic signs |
| | regional lymph node involvement | |

| | |
|---|---|
| IV | Any tumor with distant metastasis or recurrence with metastasis |
| | (including blood or bone marrow involvement) |

Exclusion and Removal Criteria:

Dogs that had previously been treated with Compound 1 or dogs that had been identified with characteristics in accordance with Table 11 below or were debilitated, suffering from disease other than MCT or injury, fractious or otherwise unsuitable for inclusion in the study, in the opinion of the Co-Investigator, were not enrolled.

TABLE 11
Exclusion Criteria

Stage IIb, III or IV MCTs
Subcutaneous MCT
Tumor < 0.1 cm³ or > 6.0 cm³
Weight of dog < 5 kg
Pregnant or lactating dogs, or dogs intended for breeding purposes
Dogs with evidence of serious systemic MCT disease
Administration of corticosteroids within 14 days prior to enrolment in
the study
Evidence of gastrointestinal bleeding or evidence of coagulopathy
Prior radiation therapy regimen or systemic chemotherapy regime for
treatment of the MCT
Life Expectancy < 12 weeks

As determined by the Co-Investigator, an enrolled dog could be removed from the study prior to study completion under the following extenuating circumstances (as re-stated from the study protocol):

i. If at any time during the study, in the opinion of the Co-Investigator, the dog's welfare is at risk due to non-response of the tumor to Compound 1 treatment or due to a serious adverse event, the Co-Investigator can elect to terminate the dog from the study (performing the necessary study procedures as described in this Protocol). Following termination, the dog's attending veterinarian can administer alternative treatment and veterinary care that they deem is appropriate and in the best interest of the dog.

ii. Owner elects to voluntarily withdraw dog from study.

iii. It is determined during the study that the dog did not actually meet the eligibility requirements (e.g. clinically significant blood results from Day 0 sampling indicate that the dog is not acceptable for study participation, etc.)

iv. Dog requires medications prohibited by the protocol.

Three dogs were removed from the study once enrolled.

Allocation:

Dogs were enrolled in the study as they presented to participating veterinary practices meeting the necessary eligibility criteria. As the study design was a dose de-escalation study, dogs were allocated to the highest dose initially (1.0 mg Compound 1 per mL). Each dose comprised a total cohort of 10 dogs participating across all clinics. Once the cohort of 10 dogs was treated at the highest dose, subsequent dogs were assigned to the 2^{nd} cohort (0.5 mg Compound 1 per mL) and dosing continued through de-escalation until completion of 7 evaluable enrolments in the 3^{rd} cohort (0.2 mg Compound 1 per mL).

Each Compound 1 preparation (1.0, 0.5, and 0.2 mg/mL) was pre-prepared in a 30% propylene glycol, 30 mM Acetate buffer pH 4.0 solution.

Dose Calculation:

Compound 1 was administered to each dog at 0.5 mL per cm³ of tumor volume as determined on the day of dosing. Digital calipers were used to measure tumor axes. MCT volume was estimated using a modified ellipsoid method ½ (length (cm)×width (cm)×depth (cm)), suggested as one of the most accurate caliper volume calculations for a palpable tumor (Faustino-Rocha et al. 2013).

Dose Preparation:

The required volume of the designated dose concentration of Compound 1 was drawn into an appropriate 1 mL or 3 mL Terumo luer lock syringe and fitted with a 23 G or 25 G needle. It was acceptable to use the drug concentrations of 0.5 mg/mL and 0.2 mg/mL directly from the refrigerator. Due to some cloudiness occurring with the 1.0 mg Compound 1 per mL at the refrigerated temperature, these vials were removed from the refrigerator at least 3 hours, but no more than 48 hours, prior to treatment to ensure that Compound 1 was totally dissolved.

Method of Dose Administration:

Study dogs were dosed according to the treatment regime detailed in Table 12 below.

TABLE 12
Treatment Regime

| | | Dose Volume* | | | |
|---|---|---|---|---|---|
| | | (mL per cm³ of | | | |
| Cohort | Drug Details | tumor) | Route | Freq. | No. |
| 1 | Compound 1: | 0.5 | Intratumoral | once | 10 |
| | 1.0 mg/mL | | | | |
| 2 | Compound 1: | 0.5 | Intratumoral | once | 10 |
| | 0.5 mg/mL | | | | |
| 3 | Compound 1: | 0.5 | Intratumoral | once | 7^{†} |
| | 0.2 mg/mL | | | | |

Study terminated at completion of cohort 3 because <3 dogs show efficacy (greater than a 70% reduction in tumor volume at 21 days).

Wherever possible, the tumor and a 2 cm border surrounding the tumor was shaved prior to treatment. To deliver the drug formulation, the needle of the dosing syringe was inserted into the tumor at the injection point and moved in a radial manner in 2- and 3-dimensions with approximately 0.1 mL of formulation delivered to each 0.2 cm³ of tumor mass. Study dogs were clinically examined at approximately 24 and 48 hours, 7, 14 and 21 days post-treatment.

TABLE 13
Schedule of Events
This schedule approximates the conduct of
activities for each enrolled dog.

| Approx. | |
|---|---|
| Study Day | Activities |
| Screening | A dog was enrolled if it met inclusion criteria (including |
| visit | confirmed diagnosis of cutaneous MCT via fine needle |
| | aspirate (FNA)). A clinical examination and tumor |
| | assessment was performed. Owners' consent was |
| | obtained for dogs presenting to the clinic that were likely to |
| | be suitable for enrolment in the study. |
| Pre- | Prophylactic treatments were given. A clinical examination |
| treatment | was performed, and a tumor assessment was recorded. |
| | Blood samples were collected for haematology, serum |
| | biochemistry, and a plasma sample for profiling drug. |
| Day 0 | Treatment was administered in accordance with Day 0 |
| | tumor volume measurement. Blood samples were collected |
| | at 0.5, 1, 2, 4 and 8 hours post treatment for plasma |
| | profiling of drug. Mandatory supportive treatments were |
| | given. Dogs were hospitalised overnight. |
| Day 1 | A clinical examination was performed, and a tumor |
| (approx. | assessment recorded. Blood sample was collected for |
| 24 h post | plasma profiling of drug. Dogs were hospitalised overnight |
| treatment) | if required. Dogs were discharged from the clinic if no |
| | adverse events were apparent. |
| Day 2 | A clinical examination was performed, and a tumor |
| (approx. | assessment recorded. Dogs held over from Day 1 were |
| 48 h post | discharged from the clinic if no adverse events |
| treatment) | were apparent. |
| Day 7 ± 1 | A clinical examination was performed, and a tumor |
| (Week 1) | assessment recorded. Blood samples were collected for |
| | haematology and serum biochemistry. Mandatory post- |
| | treatment medications were given. |
| Day 14 ± 1 | A clinical examination was performed, and a tumor |
| (Week 2) | assessment recorded. |
| Day 21 ± 2 | A clinical examination was performed, and a tumor |
| (Week 3) | assessment recorded. Blood samples were collected for |
| | haematology and serum biochemistry prior to the |
| | dog's discharge from study. |

Prophylactic and supportive medications considered for use in this study to prevent paraneoplastic MCT-induced adverse events such as Darier's sign and gastric upset included cetrizine, chlorpheniramine, omeprazole, acetylpromazine, methadone and meloxicam. Some supportive therapies were given up to 5 days post treatment (see Table 14). Additional approved supportive therapies (if required) included fentanyl (patch) or tramadol analgesia, or antibiotic (amoxicillin/clavulanic acid).

TABLE 14
Prophylactic and Supportive Medications

| Drug | | Pre-Tx | | Post-Tx | | | | | |
|---|---|---|---|---|---|---|---|---|---|
| [dose level] | Drug Class | Day −1 | Day 0 | Day 0 | Day 1 | Day 2 | Day 3 | Day 4 | Day 5 |
| Cetrizine (oral) | H1 | X | | X | X | X | X | X | X |
| 0.25 mg/kg BWt | antihistamine | | | | | | | | |
| SID | | | | | | | | | |
| Chlorpheniramine | H1 | | X | | | | | | |
| (IM) | antihistamine | | | | | | | | |
| 5 mg for ≤10 kg | | | | | | | | | |
| BWt | | | | | | | | | |
| 5 mg per 10 kg | | | | | | | | | |
| (>10 kg BWt) | | | | | | | | | |
| Omeprazole (oral) | Proton pump | X | | X | X | X | X | X | X |
| 1.0 mg/kg BWt | inhibitor | | | | | | | | |
| SID | | | | | | | | | |
| Acetylpromazine | Sedative & | | X | | | | | | |
| (SC) | antiemetic | | | | | | | | |
| 0.005-0.2 mg/kg | | | | | | | | | |
| BWt | | | | | | | | | |
| Methadone (SC) | Analgesic | | X | | | | | | |
| 0.04 mg/kg BWt | | | | | | | | | |
| Meloxicam (SC) | Anti- | | X | | | | | | |
| 0.2 mg/kg BWt | inflammatory | | | | | | | | |
| | & analgesic | | | | | | | | |
| Meloxicam (oral) | Anti- | | | | X | X | X | X | X |
| 0.1 mg/kg BWt | inflammatory | | | | | | | | |
| SID | & analgesic | | | | | | | | |

Tx = treatment;
IM = intramuscular;
SC = subcutaneous;
BWt = bodyweight;
SID = once daily dosing

Injection Site Assessments and Tumor Reactions:

Determination of efficacy was based on objective tumor measurements made according to the RECIST v.1.1 guidelines (Eisenhauer et al., 2009) using unidirectional tumor measurements.

Plasma Profile Analysis:

A single (approximate) 4 mL blood sample for plasma profiling was collected from the dogs at intervals outlined in Table 12 by venipuncture using fresh sterile needles and a lithium heparinised Vacuette®. Processing of each vacutainer commenced within 20 minutes of sample collection. If a delay to sample processing was unavoidable, then the PK sample was stored on ice until processing could occur. Plasma was harvested from vacuettes following centrifugation at approximately 1300 g-force (rcf) for 10 minutes. The plasma obtained was divided into duplicate samples with a minimum of 0.6 mL each and placed into duplicate plastic tubes (Replicate 1 and 2 samples). Vials were individually labeled with the study number, dog number, replicate, study date & day, time point and sample type. Samples were stored frozen (−10 to −30° C.) in temperature monitored freezers for analysis.

Serum Biochemistry:

Individual blood samples were collected from the dogs into gel separated plain vacuettes at intervals outlined in Table 12. Plain blood samples were submitted on ice bricks by express courier to the local IDEXX laboratory which performed detailed biochemical analysis. Analysis included creatinine, protein, albumin, globulin, alkaline phosphatase, AST, gamma GT, creatine kinase, cholesterol, magnesium, calcium, phosphate, sodium, potassium, chloride, ALT, glucose, urea, amylase, bilirubin and triglycerides.

Haematology:

Individual blood samples were collected from the dogs into anti-coagulated (EDTA and sodium citrate) vacutainers at intervals outlined in Table 12. Blood samples were then be submitted on ice bricks by express courier to the local IDEXX laboratory, which performed detailed haematological analysis. Analysis included red blood cell count, white cell count, packed cell volume, mean corpuscular volume, mean corpuscular haemoglobin, mean corpuscular haemoglobin concentration, white cell differential count, platelets, haemoglobin, reticulocytes, PT and APTT.

### Statistical Analysis

Pairwise comparison of categorical responses to treatment between treatment groups at each time point post-treatment were compared using Fisher's Exact Test. Using the RECIST classification (Eisenhauer et al., 2009), tumor data was analysed to determine the effectiveness of, and differences between, treatments. Generalised linear modeling was used to examine the combined effects of treatment dose, time post-treatment and other potentially explanatory variables. Generalised estimating equations with appropriate error structure (and controlled for repeated measurements of individuals) were used to examine categorical response to treatment and changes in tumor volume measurement. Tumor volume analysis was also controlled for pre-treatment tumor volume.

### Results

Patient Demographics and Treatment Details:

Patient demographics for the twenty seven (27) evaluable cases in the 3 cohorts are presented in Table 15.

TABLE 15
Patient demographics

| | | | | BW (kg) | | |
|---|---|---|---|---|---|---|
| | Dog | | Age | Immediately | | |
| Cohort | ID | Breed | (yrs) | Pre-treatment | Sex | Tumor Location |
| 1 | NVC- | Labrador Retriever X | 8 | 35.7 | M | Left forelimb |
| | 101 | | | | | |
| 1 | NVC- | Staffordshire Terrier | 12 | 21.4 | F | Right base of |
| | 102 | | | | | tail |
| 1 | NVC- | Labrador Retriever | 5 | 33.4 | F | Left mammary |
| | 103 | | | | | |
| 1 | NVC- | Rhodesian Ridgeback X | 8 | 24.8 | F | Left base of tail |
| | 104 | | | | | |
| 1 | TVS- | Boxer | 7 | 33.3 | F | Left thoracic |
| | 105 | | | | | wall |
| 1 | TVS- | Australian Border Collie | 8 | 21.1 | F | Right forefoot |
| | 106 | | | | | |
| 1 | GCV- | Pug | 6 | 9.0 | F | Right hindlimb |
| | 107 | | | | | |
| 1 | TVS- | Staffordshire Terrier | 6 | 22.5 | F | Left rump |
| | 108 | | | | | |
| 1 | GCV- | Labrador Retriever | 8 | 32.5 | F | Left forelimb |
| | 109 | | | | | |
| 1 | NVC- | Staffordshire Terrier | 8 | 29.1 | M | Left perianal |
| | 110 | | | | | |
| 2 | GCV- | Staffordshire Terrier X | 13 | 19.0 | F | Left hindlimb |
| | 201 | | | | | |
| 2 | TVS- | Mastiff | 2 | 20.4 | F | Vulva |
| | 202 | | | | | |
| 2 | TVS- | Maltese X | 7 | 7.4 | F | Right maxilla |
| | 203 | | | | | |
| 2 | GCV- | Siberian Husky X | 7 | 28.7 | F | Right hindlimb |
| | 204 | | | | | |
| 2 | TVS- | Staffordshire Terrier | 6 | 25.8 | F | Right flank |
| | 205 | | | | | |
| 2 | ANV- | Bull Mastiff X | 10 | 35.5 | M | Right pelvis |
| | 206 | | | | | |
| 2 | GCV- | Neopolitean Mastiff X | 6 | 38.9 | M | Right thoracic |
| | 207 | | | | | wall |
| 2 | NVC- | Staffordshire Terrier | 8 | 25.8 | M | Right shoulder |
| | 208 | | | | | |
| 2 | TVS- | Basenji | 15 | 10.4 | F | Left side axilla |
| | 209 | | | | | |
| 2 | TVS- | Staffordshire Terrier X | 8 | 31.9 | M | Periocular |
| | 210 | | | | | |
| 3 | NVC- | Staffordshire Terrier | 7 | 15.9 | F | Left thoracic |
| | 301 | | | | | wall |
| 3 | NVC- | Staffordshire Terrier | 8 | 34.9 | F | Left mammary |
| | 304 | | | | | |
| 3 | NVC- | Maltese | 12 | 9.9 | M | Right flank |
| | 305 | | | | | |
| 3 | NVC- | Boxer | 4 | 29.3 | F | Right hindlimb |
| | 306 | | | | | |
| 3 | NVC- | Labrador Retriever | 10 | 24.0 | F | Base of right ear |
| | 307 | | | | | |
| 3 | NVC- | Pharaoh Hound | 8 | 20.2 | F | Vulva |
| | 308 | | | | | |
| 3 | NVC- | Terrier | 5 | 7.5 | F | Left lateral |
| | 310 | | | | | thigh |

ID = identification;
BW = bodyweight;
F = female;
M = male;
X = cross

Median age upon study enrolment was 8.0 years (range 2-15) and median bodyweight was 24.0 kg (range 7.4-38.9). Twenty (20) females and seven (7) males were enrolled. The most common breeds represented were Staffordshire Terrier (or cross) (n=9), and Labrador Retriever (or cross) (n=4).

Calculated tumor volumes (cm³) and treatment details are summarised in Table 16.

TABLE 16
Calculated tumor volumes and treatment details

| | | | Calc | | | | |
|---|---|---|---|---|---|---|---|
| | | | tumor | | | | Amount of |
| | | | volume | | Comp 1 | Dose | Comp 1 |
| | Dog | | (cm³) | Date of | conc. | Volume | delivered |
| Cohort | ID | Breed | Day 0 | treatment | (mg/mL) | (mL) | (mg) |
| 1 | NVC- | Labrador | 2.90 | 12-Mar- | 1.0 | 1.45 | 1.45 |
| | 101 | Retriever X | | 13 | | | |
| 1 | NVC- | Staffordshire | 0.65 | 03-Jun- | 1.0 | 0.35 | 0.35 |
| | 102 | Terrier | | 13 | | | |
| 1 | NVC- | Labrador | 1.62 | 03-Jun- | 1.0 | 0.80 | 0.80 |
| | 103 | Retriever | | 13 | | | |
| 1 | NVC- | Rhodesian | 0.42 | 18-Jun- | 1.0 | 0.20 | 0.20 |
| | 104 | Ridgeback X | | 13 | | | |
| 1 | TVS- | Boxer | 0.90 | 11-Jun- | 1.0 | 0.45 | 0.45 |
| | 105 | | | 13 | | | |
| 1 | TVS- | Australian Border | 0.81 | 12-Jun- | 1.0 | 0.40 | 0.40 |
| | 106 | Collie | | 13 | | | |
| 1 | GCV- | Pug | 1.69 | 02-Jul- | 1.0 | 0.85† | 0.85 |
| | 107 | | | 13 | | | |
| 1 | TVS- | Staffordshire | 0.73 | 08-Jul- | 1.0 | 0.36¥ | 0.36 |
| | 108 | Terrier | | 13 | | | |
| 1 | GCV- | Labrador | 1.73 | 23-Jul- | 1.0 | 0.85 | 0.85 |
| | 109 | Retriever | | 13 | | | |
| 1 | NVC- | Staffordshire | 4.69 | 29-Jul- | 1.0 | 2.35 | 2.35 |
| | 110 | Terrier | | 13 | | | |
| 2 | GCV- | Staffordshire | 1.87 | 20-Aug- | 0.5 | 0.95 | 0.48 |
| | 201 | Terrier X | | 13 | | | |
| 2 | TVS- | Mastiff | 0.36 | 02-Sep- | 0.5 | 0.20 | 0.10 |
| | 202 | | | 13 | | | |
| 2 | TVS- | Maltese X | 4.03 | 04-Nov- | 0.5 | 2.00 | 1.00 |
| | 203 | | | 13 | | | |
| 2 | GCV- | Siberian Husky X | 0.36 | 11-Nov- | 0.5 | 0.20 | 0.10 |
| | 204 | | | 13 | | | |
| 2 | TVS- | Staffordshire | 0.17 | 12-Nov- | 0.5 | 0.10 | 0.05 |
| | 205 | Terrier | | 13 | | | |
| 2 | ANV- | Bull Mastiff X | 1.20 | 20-Nov- | 0.5 | 0.60 | 0.30 |
| | 206 | | | 13 | | | |
| 2 | GCV- | Neopolitean | 0.77 | 09-Dec- | 0.5 | 0.38 | 0.19 |
| | 207 | Mastiff X | | 13 | | | |
| 2 | NVC- | Staffordshire | 0.26 | 28-Jan- | 0.5 | 0.13 | 0.07 |
| | 208 | Terrier | | 14 | | | |
| 2 | TVS- | Basenji | 4.15 | 28-Jan- | 0.5 | 2.05* | 1.03 |
| | 209 | | | 14 | | | |
| 2 | TVS- | Staffordshire | 0.36 | 03-Feb- | 0.5 | 0.20 | 0.10 |
| | 210 | Terrier X | | 14 | | | |
| 3 | NVC- | Staffordshire | 0.39 | 10-Feb- | 0.2 | 0.20 | 0.04 |
| | 301 | Terrier | | 14 | | | |
| 3 | NVC- | Staffordshire | 0.16 | 02-Jun- | 0.2 | 0.08 | 0.02 |
| | 304 | Terrier | | 14 | | | |
| 3 | NVC- | Maltese | 0.51 | 14-Jul- | 0.2 | 0.30 | 0.06 |
| | 305 | | | 14 | | | |
| 3 | NVC- | Boxer | 0.66 | 28-Jul- | 0.2 | 0.35 | 0.07 |
| | 306 | | | 14 | | | |
| 3 | NVC- | Labrador | 0.43 | 18-Aug- | 0.2 | 0.22 | 0.04 |
| | 307 | Retriever | | 14 | | | |
| 3 | NVC- | Pharaoh Hound | 0.30 | 25-Aug- | 0.2 | 0.15 | 0.03 |
| | 308 | | | 14 | | | |
| 3 | NVC- | Terrier | 0.22 | 01-Sep- | 0.2 | 0.11 | 0.02 |
| | 310 | | | 14 | | | |

The median tumor volume on Day 0 (the day of treatment) was 0.66 cm³ (range 0.16-4.69). At a dose level of 0.5 mL Compound 1 per cm³ tumor, the median dose volume (mL) was 0.35 mL (range 0.08-2.35).

Body Weights:

Body weights were collected on each enrolled dog at screening, immediately pre-treatment, at approximately 24 hours, 48 hours post-treatment, and at 7(+1) days, 14(+1) days and 21(+2) days post treatment. The percent bodyweight change is calculated as the difference between immediate pre-treatment and Day 21 (+2) post-treatment values. A positive value indicates weight gain, a negative value indicates weight loss.

In cohort 1 (1.0 mg Compound 1 per mL), 6 out of 10 dogs gained weight during the course of the trial. One dog (NVC-102) gained 15% body weight. Of those dogs (3) that lost weight, 3.9% weight loss was the largest recorded value (dog NVC-103). In cohort 2 (0.5 mg Compound 1 per mL), 9 out of 10 dogs lost body weight during the course of the trial. The largest recorded loss was 5.6% (dog GCV-204). In cohort 3 (0.2 mg Compound 1 per mL), 3 out of 7 dogs gained weight, and 4 out of 7 dogs lost weight during the course of the trial. The largest recorded weight loss was 9.2% (dog-NVC-304).

Clinical Examinations:

Clinical parameters such as rectal temperature (° C.), heart rate (HR) per minute, respiratory rate (RR) per minute, capillary refill time (CRT) (sec), systolic and diastolic blood pressure (mmHg), and mucosal colour were collected on each enrolled dog at screening, immediately pre-treatment, at approximately 24 hours, 48 hours post-treatment, and at 7(±1), 14(±1) and 21(±2) days post treatment. With each parameter evaluated for trends over time, and in context with all other contemporaneously collected parameters, the clinical parameter data is deemed unremarkable for all trial dogs.

Dogs were evaluated for adverse events (AEs) on Days 0 (pre-treatment), 1, 2, 7(±1), 14(±1) and 21(±2) as well as interim timepoints if needed. An AE was defined as any unfavorable and unintended sign, symptom, or disease temporally associated with the use of Compound 1, whether or not related to the product (VCOG-CTCAE 2011).

In this study, 64 AEs were identified in 15 event types (see Table 16). The majority of identified AEs (52) were represented by the ‘mild’ VCOG-CTCAE classification Grade 1 with no intervention required. A level of minimal medical intervention (antimicrobial e.g. amoxicillin/clavulanic acid) or additional analgesic therapy (e.g. fentanyl patch or tramadol) was required in some cases (12 AEs) during the post treatment monitoring period. The incidence rates of each reported AE and associated VCOG-CTCAE grade classification are summarised in Table 17. These findings were all transient in nature.

TABLE 17
Incidence rates and VCOG-CTCAE classification
of adverse events (AEs)

| | All patients (n = 27) | | |
|---|---|---|---|
| | | | VCOG- |
| | | | CTCAE |
| | No. of | Incidence | Grade |
| Event type (medical intervention) | events | (%) | classification |
| Local swelling at treatment site* | 22 | 81.5 | 1 |
| Pain associated with treatment site* | 4 | 14.8 | 1 |
| Pain associated with treatment site* | 6 | 22.2 | 2 |
| (oral analgesic) | | | |
| Transient tachypnoea | 14 | 51.9 | 1 |
| Transient lethargy | 5 | 18.5 | 1 |
| Transient tachycardia | 2 | 7.4 | 1 |
| Bilateral otitis externa (oral | 1 | 3.7 | 2 |
| antibiotic) | | | |
| Transient elevated blood pressure | 1 | 3.7 | 1 |
| reading | | | |
| Transient salivation | 1 | 3.7 | 1 |
| Emesis | 2 | 7.4 | 1 |
| Flatulence (oral antibiotic) | 1 | 3.7 | 2 |
| Seborrhoea (oral antibiotic) | 1 | 3.7 | 2 |
| Corneal ulcer (antibiotic ointment | 1 | 3.7 | 2 |
| and oral analgesic) | | | |
| Wound sinus | 1 | 3.7 | 1 |
| Wound infection (oral antibiotic) | 2 | 7.4 | 2 |
| Total no. of AEs | 64 | | |

*Not an unexpected outcome due to the mechanism of action of Compound 1

Injection Site Assessments and Tumor Reactions:

MCT measurements were recorded using digital calipers. Measurements were taken pre-treatment and at 7(±1), 14(±1) and 21(±2) days post treatment. The length (cm) was designated the longest measurable axis; the width (cm) was the tumor at the widest point perpendicular to the length; gentle manipulation of the MCT to measure the depth (cm) using the calipers was considered acceptable. Determination of efficacy was based on objective tumor measurements made according to the RECIST v.1.1 guideline using the longest unidirectional measurement (Eisenhauer et al. 2009) recorded at each timepoint. RECIST criteria were only applied to target lesions as Compound 1 is not intended as a systemic therapy. Response to therapy was defined as complete response (CR, resolution of the target lesion), partial response (PR, at least 30% decrease in the longest diameter of target lesion), stable disease (SD, decrease in the target lesion of less than 30% or increase of the target lesion less than 20%) or progressive disease (PD, greater than 20% increase in the target lesion). RECIST v.1.1 results are summarized below in Table 18.

TABLE 18
Response to Therapy

| | | | Calculated | Drug | | |
|---|---|---|---|---|---|---|
| | | | tumor | Concentration | | |
| | | | volume | (mg | Day of | |
| | | | (cm³) | Compound | RECIST | RECIST |
| Cohort | Dog ID | Breed | Day 0 | 1/mL) | assessment | classification |
| 1 | NVC- | Labrador Retriever X | 2.90 | 1.0 | 21 | CR |
| | 101 | | | | | |
| 1 | NVC- | Staffordshire | 0.65 | 1.0 | 21 | CR |
| | 102 | Terrier | | | | |
| 1 | NVC- | Labrador Retriever | 1.62 | 1.0 | 21 | CR |
| | 103 | | | | | |
| 1 | NVC- | Rhodesian | 0.42 | 1.0 | 21 | CR |
| | 104 | Ridgeback X | | | | |
| 1 | TVS- | Boxer | 0.90 | 1.0 | 21 | CR |
| | 105 | | | | | |
| 1 | TVS- | Australian Border | 0.81 | 1.0 | 21 | CR |
| | 106 | Collie | | | | |
| 1 | GCV- | Pug | 1.69 | 1.0 | 21 | SD |
| | 209^{E} | | | | | |
| 1 | TVS- | Staffordshire | 0.73 | 1.0 | 21 | CR |
| | 108 | Terrier | | | | |
| 1 | GCV- | Labrador Retriever | 1.73 | 1.0 | 21 | CR |
| | 109 | | | | | |
| 1 | NVC- | Staffordshire | 4.69 | 1.0 | 21 | CR |
| | 110 | Terrier | | | | |
| 2 | GCV- | Staffordshire | 1.87 | 0.5 | 21 | CR |
| | 201 | Terrier X | | | | |
| 2 | TVS- | Mastiff | 0.36 | 0.5 | 21 | SD |
| | 202 | | | | | |
| 2 | TVS- | Maltese X | 4.03 | 0.5 | 21 | CR |
| | 203 | | | | | |
| 2 | GCV- | Siberian Husky X | 0.36 | 0.5 | 21 | CR |
| | 204 | | | | | |
| 2 | TVS- | Staffordshire | 0.17 | 0.5 | 21 | CR |
| | 205 | Terrier | | | | |
| 2 | ANV- | Bull Mastiff X | 1.20 | 0.5 | 21 | SD |
| | 206 | | | | | |
| 2 | GCV- | Neopolitean | 0.77 | 0.5 | 21 | CR |
| | 207 | Mastiff X | | | | |
| 2 | NVC- | Staffordshire | 0.26 | 0.5 | 21 | PR |
| | 208 | Terrier | | | | |
| 2 | TVS- | Basenji | 4.15 | 0.5 | 21 | SD |
| | 209 | | | | | |
| 2 | TVS- | Staffordshire | 0.36 | 0.5 | 21 | PD |
| | 210 | Terrier X | | | | |
| 3 | NVC- | Staffordshire | 0.39 | 0.2 | 21 | SD |
| | 301 | Terrier | | | | |
| 3 | NVC- | Staffordshire | 0.16 | 0.2 | 21 | SD |
| | 304 | Terrier | | | | |
| 3 | NVC- | Maltese | 0.51 | 0.2 | 21 | CR |
| | 305 | | | | | |
| 3 | NVC- | Boxer | 0.66 | 0.2 | 21 | PR |
| | 306 | | | | | |
| 3 | NVC- | Labrador Retriever | 0.43 | 0.2 | 21 | SD |
| | 307 | | | | | |
| 3 | NVC- | Pharaoh Hound | 0.30 | 0.2 | 21 | SD |
| | 308 | | | | | |
| 3 | NVC- | Terrier | 0.22 | 0.2 | 21 | CR |
| | 310 | | | | | |

ID = identification;
CR = complete response;
PR = partial response;
SD = stable disease;
PD = progressive disease

Of the 10 dogs treated in cohort 1 (1.0 mg Compound 1 per mL), 9 experienced complete response (CR) and 1 experienced stable disease (SD). The dog experiencing SD (GCV-107; Pug, tumor size 1.69 cm³ on Day 0) had a unidirectional tumor measurement (length) decrease of 23% by Day 21, and a total tumor volume reduction of 36% (modified ellipsoidal calculation). This dog was treated by surgical removal of the residual ulcerative tumor with no mitotic nuclei identified in post-surgical biopsy material. With dose de-escalation, of the 10 dogs treated in cohort 2 (0.5 mg Compound 1 per mL), 5 experienced complete response (CR), 1 partial response (PR), 3 stable disease (SD), and 1 progressive disease (PD). Of the 7 dogs treated in cohort 3 (0.2 mg EBC-46 per mL), 2 experienced complete response (CR), 1 partial response (PR), and 4 stable disease (SD).

In order to confirm the effectiveness of treatment, statistical analysis was conducted on the efficacy data from the 27 trialed dogs. RECIST scoring (% longest tumor diameter reduction at Day 21) and RECIST classification were introduced to the analysis. Data indicated that dogs treated with the highest dose level of 1.0 mg Compound 1 per mL (cohort 1) had the highest proportion of subjects attaining the RECIST classification of ‘complete response’ (CR) (p<0.05). Consequently, the 1.0 mg/mL formulation is now selected as the therapeutic concentration of the drug.

Plasma Profile Analysis:

Blood samples (approximately 4 mL) for measurement of plasma drug levels were collected at pre-dose on Day 0, and then at approximately 0.5, 1, 2, 4, 8 and 24 hours post-dose. The pharmacokinetic evaluation was performed on 26 dogs, being 9 in cohort 1, 10 in cohort 2, and 7 in cohort 3. Four (4) dogs were excluded from the analysis. No samples were available for dog NVC-104 (cohort 1) due to the aggressive behaviour of the dog. Dogs CSV-302, GCV-303 and GCV-309 (all cohort 3) were also excluded

For each remaining dog, individual plasma concentration-time data of Compound 1 were used for the calculation of Cₘₐₓ, Tₘₐₓ, AUCₗₐₛₜ, AUC_{inf}, λ_{z} and T_{1/2} calculated as T_{1/2}=ln(2)/λ_{z}. Pharmacokinetic parameters of Compound 1 were calculated using Phoenix WinNonlin Version 6.3 and tabulated by dog and summarised by dose cohort. Descriptive statistics (e.g. mean, standard deviation) were calculated using Microsoft Excel 2007, which was also used to prepare graphs of the pharmacokinetic data.

In this pharmacokinetic study, it is recognized and accepted that Cₘₐₓ and Tₘₐₓ are founded ostensibly on the first timepoint of sample collection (0.5 h). If dog GCV-107 (dose leakage) (cohort 1) and TVS-203 (statistical outlier) (cohort 2) are omitted from the data set, the regression model of Cₘax (ng/mL) vs. normalised body weights (mg Compound 1 per kg) showed a consistent trend (r²=0.94). In omitting the data set for the same two dogs, the regression model of AUCₗₐₛₜ vs. normalised body weights (mg Compound 1 per kg) appeared approximately proportional to dose normalised by bodyweight (r²=0.59).

Serum Biochemistry:

The observations are fairly unremarkable with the exception of dog GCV-204 (cohort 2) with slightly elevated AST (asparate transaminase) and elevated CK (creatine kinase) readings post treatment; and dog NVC-307 (cohort 3) with transiently elevated AST and ALT (alanine aminotransferase) readings post treatment. The observed clinical parameters for dog GCV-204 and dog NVC-307 appeared normal and were relatively consistent and predictable both pre and post treatment. Neither dog was reported as unwell by attending veterinarians. Indeed, throughout the period of elevated AST and/or ALT readings (Days 7, 11 and 14), dog NVC-307 was reported to be in good health. By Day 11, the AST reading had returned to normal range, and the ALT reading had reduced significantly. The ALT level had turned to normal range by Day 21 post treatment.

Haematology:

The observations are also fairly unremarkable. The exceptions include slightly elevated reticulocyte counts observed in 7 dogs post treatment (NVC-102, TVS-106, GCV-107 (cohort 1); TVS-203, GCV-207, NVC-208 (cohort 2); NVC-306 (cohort 3)). In no dog were elevated reticulocyte readings associated with abnormal packed cell volume measures (Ht, haematocrit). No dog exhibited anaemia during the course of the study. Five (5) dogs were also observed to have slightly lower platelet counts in the post treatment period (TVS-106, TVS-108 (cohort 1); TVS-202, TVS-205, GCV-207 (cohort 2)). However for all these dogs, the platelets were described as ‘clumping’ which can falsely lead to lower counts on automated analysers (Koplitz et al., 2001). No dog exhibited a bleeding disorder during the course of the study.

### Example 3: Long Term Follow Up of Cohort 1 Dogs

The dogs of cohort 1 in Example 2, treated with 1.0 mg Compound 1 per mL, were followed up post-treatment.

Dog GCV-209 and dog NVC-110 did not participate in case follow up in the ensuing 12 month period. Dog GCV-209 was treated by surgical removal of residual tumor soon after study completion (Day 21). For dog NVC-110, tumor recurrence was reported on Day 63 (42 days or 6 weeks after study completion) and the owner opted for surgery (see Table 19, Nil=no tumor recurrence).

TABLE 19
Case follow up post treatment

| | Month of case follow-up post treatment | | | | | | | | | | | | | |
|---|---|---|---|---|---|---|---|---|---|---|---|---|---|---|
| Animal Code | 1 | 2 | 3 | 4 | 5 | 6 | 7 | 8 | 9 | 10 | 11 | 12 | 13 | 14 |

| | | | | | | |
|---|---|---|---|---|---|---|
| NVC-101 | | | | Nil | | Nil |
| NVC-102 | | Nil | | | Nil | |
| NVC-103 | | Nil | Nil | | Nil | |
| NVC-104 | | Nil | | | Nil | |
| TVS-105 | | | | | Nil | |
| TVS-106 | Nil | | | | Nil | |

| | |
|---|---|
| GCV-209 | Opted for Surgery |

| | | | |
|---|---|---|---|
| TVS-108 | | | Nil |
| GCV-109 | Nil | Nil | |

| | |
|---|---|
| NVC-110 | Opted for Surgery |

Of the 8 dogs eligible for case follow up, all had an examination for tumor recurrence out to a range of 11.9 months (357 days; GCV-109) to 15.0 months (449 days; NVC-101) post treatment. Five (5) dogs also received an interim examination between 6 to 9 months post-treatment. Dog NVC-103 was examined on 3 occasions. No tumor recurrence was found at the treatment site in any examined animal during the post treatment examination period.

### Example 4: Long Term Follow Up in Dogs

The dogs of in Example 1, treated with 1.0 mg Compound 1 per mL in 40% propylene glycol solution (0.4 mL/cm³ tumor), were followed up post-treatment.

Dogs C03-T-04 and C03-T-07 did not participate in case follow up in the ensuing 12 month period. The attending veterinarian advised dog C03-T-04 enter a new treatment program for residual tissue mass treatment due to a putative immune deficiency, and dog C03-T-07 died on Day 4 post treatment.

TABLE 20
Case follow up results post treatment

| | Repeat | | | | | | | | | | | | |
|---|---|---|---|---|---|---|---|---|---|---|---|---|---|
| Animal | treatment | Approx. month of case follow-up: time after last treatment (months) | | | | | | | | | | | |
| Code | required | 1 | 2 | 3 | 4 | 5 | 6 | 7 | 8 | 9 | 10 | 11 | 12 |
| C03-N-01 | Nil | | | | | | TR | Opted for surgery | | | | | |
| C03-N-02 | Nil | | | | | | NTR | | | | | | NTR |
| (C03-N-03) | Day 28 | | | | NTR | | | | | | | | NTR |
| (C03-T-01) | Day 28 | | NTR | | | NTR | | NTR | | | | NTR | |
| C03-T-02 | Nil | | | NTR | | | NTR | | | NTR | | | TR |
| (C03-T-03) | Day 29 | | NTR | NTR | | NTR | | | | | | | NTR |
| C03-T-05 | Nil | | | NTR | | | NTR | | | NTR | | | NTR |

| C03-T-06 | Nil | NTR | NTR | NTR | Euthanased | |
|---|---|---|---|---|---|---|
| C03-T-08 | Nil | NTR | NTR | NTR | | NTR |
| C03-T-09 | Nil | NTR | NTR | NTR | | NTR |

parenthesis on animal code = residual neoplastic tissue identified on Days 28-29
NTR = no tumor recurrence;
TR = tumor recurrence;

No Residual Tumor Mass or Recurrence.

Of the ten (10) dogs available for follow up assessment, five (5) were treated successfully with a single treatment of Compound 1 injection and had no tumor recurrence within the duration of assessment. Dog C03-T-06 remained tumor free until approx. 8 months (265 days) post treatment when it was euthanased as a result of longstanding diabetes and kidney failure.

Residual Tumor Mass.

Three (3) dogs (C03-N-03, C03-T-01 and C03-T-03) were identified with residual tumor mass on Days 28-29 post treatment and were retreated accordingly with Compound 1 injection. None of these dogs exhibited tumor recurrence post-repeat treatment when examined out to approx. 12 months (389 days), 11 months (339 days) and 11 months (346 days) respectively.

Tumor Recurrence.

Dog C03-N-01 was identified with tumor recurrence at approx. 6 months (196 days) post treatment and although retreatment was presented as a valid alternative, the owner(s) opted for surgery. Dog C03-T-02 was identified with tumor recurrence at approx. 12 months (359 days) post treatment. Subsequent treatment was conducted outside the conduct of this follow up study and is not reported.

### Example 5: Combination Therapy, Compound 1 and Oral Corticosteroid

This study investigates the efficacy and safety of Compound 1 injection for MCT treatment in the presence of corticosteroids administered under defined clinical situations.

### Inclusion Criteria:

Dogs were selected for the study if they met the criteria in Table 1 (Example 1) but where the dog has at least one MCT.

### Exclusion Criteria:

Dogs that were debilitated, suffering from disease other than MCT or injury, fractious or otherwise unsuitable for inclusion in the study, in the opinion of the attending veterinarian, were not enrolled. Animal enrolment details are provided in Table 21.

TABLE 21
Animal Enrolment Details:

| | |
|---|---|
| Species | Canis lupus familiaris (dog) |
| Number | 16† |
| Bodyweight | 6.4-41.2 kg (median 22.9 kg) |
| Sex | 14 neutered females and 2 neutered males |
| Age | 2-12 yrs (median 8 yrs) |
| Source | Client owned |
| Identification method | Enrolment no. |

†One dog was treated by a single dose of Compound 1 on three separate occasions for 3 different cutaneous MCT. In total, 13 cutaneous MCT and 5 subcutaneous MCT were treated.

Removal of Animals from Assessment:

Dog C05-V-02 was removed from the study on Day 21 due to progressive disease of a vulvar tumor.

TABLE 22
Treatment regime

| | Dose Volume | | | No. of |
|---|---|---|---|---|
| Drug Details | (mL/cm³ of tumor) | Route | Frequency | animals |
| Compound 1 | 0.4 | Intratumoral | once | 16 |
| injection | | | | |
| 1.0 mg/mL | | | | |

Wherever possible, the tumor and a 2 cm border surrounding the tumor was shaved prior to treatment. To deliver the drug, the needle of the dosing syringe was inserted into the tumor at the injection point and moved in a radial manner in 2- and 3-dimensions with drug delivered to the tumor mass. Study dogs were clinically examined at 2-4 and 24 hours, and 7 (+2), 14 (+2) and 28 (+2) days post-treatment (and unscheduled visits at attending veterinarian discretion).

### Prophylactic and Supportive Medications:

Each supervising veterinarian was given latitude to provide and record prophylactic and supportive medications as deemed appropriate for managing potential paraneoplastic events and tumor site necrosis following Compound 1 treatment. However, prednisolone and antihistamines were used as co-therapy. Prednisolone therapy commenced on Day −1 and continued for a further 7 days. H1 and H2 antihistamine therapy (chlorpheniramine and famotidine respectively) commenced on the morning of drug treatment and continued for a further 6 days (see Table 23).

TABLE 23
Co-therapy treatment regime

| Drug | Treatment Regime |
|---|---|
| Prednisolone | 0.5 mg/kg BW b.i.d. for 5 days (Day −1 to Day 3) |
| | then, 0.5 mg/kg BW s.i.d. (am) for 3 days |
| | (Day 4 to Day 6) |
| Chlorpheniramine | 0.25-0.5 mg/kg BW per os b.i.d. for 7 days |
| | (Day 0 to Day 6) |
| Famotidine | 0.5 mg/kg BW per os b.i.d. for 7 days (Day 0 to |
| | Day 6) |

per os = by mouth;
BW = bodyweight;
s.i.d. (semel in die) = once per day;
b.i.d.(bis in die) = twice per day

A full schedule of supportive therapies is given in table 24.

TABLE 24
Prophylactic and Supportive Medications

| | | | | Case ID | | | | | | | | | | | |
|---|---|---|---|---|---|---|---|---|---|---|---|---|---|---|---|
| Prophylactic and Supportive medications | | | | Co5- | Co5- | Co5- | Co5- | Co5- | Co5- | Co5- | Co5- | Co5- | Co5- | Co5- | Co5- |
| | Chemical | Trade name | Delivery | T-01 | T-02 | T-03 | T-04 | T-05 | T-07 | T-08 | T-09 | T-12 | T-13 | T-14 | T-15 |
| Pre- | Prednisolone | MACROLONE | PO | X | X | X | X | | X | X | X | X | X | X | X |
| Tx | Prednisolone | PRED-X | PO | | | | | X | | | | | | | |
| | Triamcinolone | TRIAMOLONE- | IT | | | | | | | | | | | | |
| | | FORTE | | | | | | | | | | | | | |
| | Vitamins/naturals | PRODUCT B | PO | | | | | | X | | | | | | |
| Post- | Prednisolone | MACROLONE | PO | X | X | X | X | | X | X | X | X | X | X | X |
| Tx | Prednisolone | PRED-X | PO | | | | | X | | | | | | | |
| | Chlorpheniramine | IRAMINE | PO | X | X | X | X | X | X | X | X | X | X | X | X |
| | Famotidine | FAMOTIDINE | PO | X | X | X | X | X | X | X | X | X | X | X | X |
| | Tramadol | TRAMADOL | PO | X | X | X | X | X | X | X | X | X | X | X | X |
| | Stilboestrol | STILBOESTROL | PO | X | | | | | | | | | | | |
| | Maropitant citrate | CERENIA | SC | | | | X | | | | | | | | |
| | Methadone | METHONE | SC | | | | X | X | | | X | | | | |
| | Amoxycillin/clavulanic | NOROCLAV | SC | | | | X | | | | | | | | |
| | acid | | | | | | | | | | | | | | |
| | Amoxycillin/clavulanic | AMOXYCLAV | PO | | | | X | | X | | | | | | |
| | acid | | | | | | | | | | | | | | |
| | IV fluids | HARTMANNS | IV | | | | X | | | | | | | | |
| | Imidacloprid/permethrin | ADVANTIX | TP | | | | | X | | | | | | | |
| | Liquid Gel enema | MICROLAX | PR | | | | | | | | | | | | |
| | Meloxicam | METACAM | PO | | | | | | | | | | | | |
| | Metronidazole | METOGYL | PO | | | | | | | | | | | | |
| | Carprofen | RIMADYL | PO | | | | | | | | X | | | | |
| | Vitamins/naturals | PRODUCT B | PO | | | | | | X | | | | | | |
| | Cephalexin | RILEXINE | PO | | | | | | | | X | | | | |
| | Promethazine | PHENERGAN | PO | | | | | | | | X | | | | |
| | Chlorhxidine/miconazole | MALASEB | TP | | | | | | | | | | | X | |
| | Miconazole | DEKARARIN | TP | | | | | | | | | | | X | |

Case ID

| | | | | | | |
|---|---|---|---|---|---|---|
| Prophylactic and Supportive medications | Co5- | Co5- | Co5- | Co5- | Co5- | Co5- |

| | Chemical | Trade name | Delivery | T-17 | T-18 | T-19 | V-01 | V-02 | N-01 |
|---|---|---|---|---|---|---|---|---|---|
| Pre-Tx | Prednisolone | MACROLONE | PO | | | | X | X | |
| | Prednisolone | PRED-X | PO | X | X | X | | | X |
| | Triamcinolone | TRIAMOLONE- | IT | | | | | | |
| | | FORTE | | | | | | | |
| | Vitamins/naturals | PRODUCT B | PO | | | | | | X |
| Post-Tx | Prednisolone | MACROLONE | PO | | | | X | X | |
| | Prednisolone | PRED-X | PO | X | X | X | | | X |
| | Chlorpheniramine | IRAMINE | PO | X | X | X | X | X | X |
| | Famotidine | FAMOTIDINE | PO | X | X | X | X | X | X |
| | Tramadol | TRAMADOL | PO | X | X | X | | X | X |
| | Stilboestrol | STILBOESTROL | PO | | | | | | |
| | Maropitant citrate | CERENIA | SC | | | | | | |
| | Methadone | METHONE | SC | X | | | | | |
| | Amoxycillin/clavulanic | NOROCLAV | SC | X | | | | | |
| | acid | | | | | | | | |
| | Amoxycillin/clavulanic | AMOXYCLAV | PO | X | | | | | |
| | acid | | | | | | | | |
| | IV fluids | HARTMANNS | IV | X | | | | | |
| | Imidacloprid/permethrin | ADVANTIX | TP | | | | | | |
| | Liquid Gel enema | MICROLAX | PR | | | | | | |
| | Meloxicam | METACAM | PO | X | | | | | |
| | Metronidazole | METOGYL | PO | | | | | | |
| | Carprofen | RIMADYL | PO | | | | | | |
| | Vitamins/naturals | PRODUCT B | PO | | | | | | |
| | Cephalexin | RILEXINE | PO | | | | | | |
| | Promethazine | PHENERGAN | PO | | | | | | |
| | Chlorhxidine/miconazole | MALASEB | TP | | | | | | |
| | Miconazole | DEKARARIN | TP | | | | | | |

ID = identification;
Tx = treatment;
IV = intravenous;
IT = intratumoural;
PO = per os (by mouth);
PR = per rectum;
SC = subcutaneous;
TP = topical

The schedule in Table 25 approximates the conduct of activities for each enrolled dog. All scheduled and unscheduled events during the study were recorded.

TABLE 25
Summary of study activities

| Approx. | |
|---|---|
| Study | |
| Day | Activities |
| Screening | A dog was enrolled if it met inclusion criteria (including |
| visit | confirmed diagnosis of an MCT via FNA. A clinical |
| | examination and tumor assessment was performed. |
| Pre- | Owners' consent was obtained for dogs presenting to the |
| treatment | clinic that were likely to be suitable for enrolment in |
| | the study (before treatment). Prophylactic medications as |
| | deemed appropriate by the supervising veterinarian |
| | were provided. |
| Day −1 | Each dog commenced treatment with oral prednisolone. |
| Day 0 | Treatment was administered in accordance with the Day 0 |
| | tumor volume measurement. Commenced treatment with |
| | H1 and H2 antihistamines. Additional supportive |
| | medications as deemed appropriate by the supervising |
| | veterinarian were prescribed. Dogs were either hospitalised |
| | overnight or discharged from hospital into client care |
| | (if condition was stable). |
| Day 1 | A clinical examination was performed. |
| Day 6 | Cessation of co-therapy treatment with prednisolone, |
| | chlorpheniramine and famotidine. |
| Day 7 ± 2 | A clinical examination was performed, a tumor assessment |
| | recorded. |
| Day 14 ± 2 | A clinical examination was performed, a tumor assessment |
| | recorded. |
| Day 28 ± 2 | A clinical examination was performed, a tumor assessment |
| | recorded. |

### Efficacy Evaluation:

Patient demographics for the eighteen (18) evaluable cases are presented in Table 26. One (1) dog had three (3) cutaneous MCT that were treated by a single dose Compound 1 injection on three (3) separate occasions. On each occasion of treatment, the dog was given a new case identification (Case IDs C05-T-12, -13, and -15) and the tumors treated at intervals of at least 28 days.

TABLE 26
Patient demographics

| | | | Bodyweight | | | |
|---|---|---|---|---|---|---|
| | | Age | (kg) (Day | | | |
| Case ID | Breed | (yrs) | 0) | Sex | Tumor Type | Tumor location |
| C05-T-01 | Boxer | 7 | 30.0 | FN | Cut MCT | Right side vulva |
| C05-T-02 | Bull x Arab | 5 | 41.2 | FN | Subcut MCT | Right craniomedial stifle |
| C05-T-03 | Cross breed | 11 | 39.0 | FN | Cut MCT | Left axilla |
| C05-T-04 | Cattle dog x | 9 | 31.2 | FN | Cut MCT | Left side ventral abdomen |
| C05-T-05 | Shi Tzu | 8 | 6.4 | FN | Subcut MCT | Left dorsal forefoot |
| C05-T-07 | Staffordshire Terrier | 7 | 21.4 | FN | Cut MCT | Right flank |
| C05-T-08 | Bull Mastiff x | 2 | 37.6 | FN | Cut MCT | Left medial thigh (above stifle) |
| C05-T-09 | German Shepherd X | 2 | 33.9 | FN | Cut MCT | Left ventral abdomen |
| C05-T-12 | Staffordshire Terrier | 8 | 21.9 | FN | Cut MCT | Right dorsal trunk |
| C05-T-13 | | | 21.1 | | Cut MCT | Behind left shoulder |
| C05-T-15 | | | 22.1 | | Cut MCT | Upper back, left side |
| C05-T-14 | Boxer | 8 | 31.5 | MN | Cut MCT | Behind left shoulder |
| C05-T-17 | Maltese Terrier | 12 | 6.4 | FN | Subcut MCT | Left hind leg, caudolateral |
| C05-T-18 | Jack Russell Terrier | 12 | 8.7 | FN | Subcut MCT | Left ventral flank |
| C05-T-19 | Jack Russell Terrier | 7 | 6.5 | FN | Cut MCT | Right shoulder |
| C05-V-01 | Boxer | 4 | 32.3 | MN | Cut MCT | Left ventral neck |
| C05-V-02 | Staffordshire Terrier | 12 | 20.0 | FN | Cut MCT | Right vulvar flap |
| C05-N-01 | Crossbred Terrier | 10.5 | 23.7 | FN | Subcut MCT | Right rump |

ID = identification;
X = cross;
BW = bodyweight;
FN = female neutered;
FE = female entire;
MN = male neutered;
ME = male entire;
MCT = mast cell tumor;
Cut = cutaneous;
Subcut = subcutaneous.

Median age upon study enrolment was 8.0 years (range 2-12) and median bodyweight was 22.9 kg (range 6.4-41.2). Fourteen (14) females and two (2) males were enrolled. All dogs were neutered. The most common breeds represented were Staffordshire Terriers (n=3) and Boxers (n=3). Calculated tumor volumes (cm³) and treatment details are summarised in Table 27.

TABLE 27
Calculated tumor volumes and treatment details

| | | Calculated | | | | Comp 1 |
|---|---|---|---|---|---|---|
| | | tumor | | | | Dose |
| | | volume | | Comp 1 | Dose | Level |
| | | (cm³) | Date of | Concentration | Volume | (% v/v |
| Dog ID | Breed | Day 0 | treatment | (mg/mL) | (mL) | tumor) |
| C05-T-01 | Boxer | 0.36 | 27-Mar-15 | 1.0 | 0.14 | 39 |
| C05-T-02 | Bull x Arab | 1.37 | 27-Mar-15 | 1.0 | 0.54 | 39 |
| C05-T-03 | Cross breed | 0.10 | 02-Apr-15 | 1.0 | 0.10 | 100 |
| C05-T-04 | Cattle dog x | 0.30 | 08-Apr-15 | 1.0 | 0.12 | 40 |
| C05-T-05 | Shi Tzu | 0.29 | 08-Apr-15 | 1.0 | 0.11 | 38 |
| C05-T-07 | Staffordshire Terrier | 0.08 | 09-Apr-15 | 1.0 | 0.10 | 125 |
| C05-T-08 | Bull Mastiff x | 0.24 | 15-Apr-15 | 1.0 | 0.10 | 42 |
| C05-T-09 | German Shepherd X | 1.32 | 15-Apr-15 | 1.0 | 0.53 | 40 |
| C05-T-12 | Staffordshire Terrier | 0.72 | 17-Jun-15 | 1.0 | 0.29 | 40 |
| C05-T-13 | | 0.23 | 15-Jul-15 | 1.0 | 0.10 | 43 |
| C05-T-15 | | 0.14 | 13-Aug-15 | 1.0 | 0.10 | 71 |
| C05-T-14 | Boxer | 0.20 | 18-Jul-15 | 1.0 | 0.10 | 50 |
| C05-T-17 | Maltese Terrier | 2.97 | 20-Aug-15 | 1.0 | 1.20 | 40 |
| C05-T-18 | Jack Russell Terrier | 0.81 | 26-Aug-15 | 1.0 | 0.32 | 40 |
| C05-T-19 | Jack Russell Terrier | 0.31 | 26-Aug-15 | 1.0 | 0.13 | 42 |
| C05-V-01 | Boxer | 0.40 | 30-Mar-15 | 1.0 | 0.20 | 50 |
| C05-V-02 | Staffordshire Terrier | 4.97 | 13-Aug-15 | 1.0 | 2.00 | 40 |
| C05-N-01 | Crossbred Terrier | 3.90 | 25-Aug-15 | 1.0 | 1.56 | 40 |

ID = identification;
x = cross;
*C05-T-07 received an ‘immune booster’ during the course of the study. This medication consists of vitamins and natural plant extracts. It is unlikely to have affected the outcome of the trial.

The median tumor volume on Day 0 (the day of treatment) was 0.3 cm³ (range 0.08-4.97). The median dose volume was 0.1 mL (range 0.1-2.0). The vast majority of cases (13/18) received a dose level that approximated 0.4 mL drug per cm³ tumor (40% v/v). Minor dose level variation (e.g. C05-T-14 (50% v/v), C05-T-15 (71% v/v), and C05-V-01 (50% v/v)) was due to the attending veterinarian selecting the nearest (or next) incremental 0.1 mL graduated mark on the syringe. Significant dose level variation was seen in two cases (C05-T-03 (100% v/v) and C05-T-07 (125% v/v)).

RECIST v.1.1 results are summarised in Table 28 which also includes a measure of neoadjuvant prednisolone action (potential tumor shrinkage) between screening and immediate pre-treatment tumor evaluation. Sixteen (16) dogs and eighteen (18) tumors were treated. Raw data for RECIST assessment was collected on Day 0 (immediately pre-treatment) and on Day 28 for fifteen (15) cases. Of these fifteen (15) cases, thirteen (13) experienced complete response (CR), 1 experienced stable disease (SD), and 1 experienced progressive disease (PD). With the remaining three (3) cases (C05-T-18, C05-V-01, and C05-V-02), the dogs were not available on Day 28 for RECIST assessment. For dog C05-T-18, the injection site was measured as CR on Days 21 and 37. The assumption is made that Day 28 would also have resulted in a CR RECIST result. For dog C05-V-01, the injection site was measured as PR on Days 21 and 37 (55% and 60% RECIST reduction respectively). The assumption is made that Day 28 would still have resulted in a partial response (PR) RECIST result. For dog C05-V-01, due to the progressive nature of disease, the animal was removed from study on Day 21. In short, a total of fourteen (14) out of eighteen (18) drug treated cases (78%) resulted in a CR RECIST result. Dogs that did not have a CR RECIST result are outlined in Table 29.

TABLE 28
Response to therapy

| | | | | Tumor response | | | |
|---|---|---|---|---|---|---|---|
| | | | | following 24 h | | | |
| | | | | prednisolone | | | |
| | | | | administration | | | |
| | | | | (screening to | | | |
| | | | | immediate pre- | | | |
| | | Calculated | | treatment) | | Efficacy assessment | |
| | | tumor | Dose | | Tumor | Day of | |
| | | volume | Volume | RECIST | vol (cm³) | RECIST | RECIST |
| Case ID | Breed | (cm³) Day 0 | (mL) | reduction | reduction | assessment(s) | classification |
| C05-T-01 | Boxer | 0.36 | 0.14 | −5% | 23% | 28 | CR |
| C05-T-02 | Bull x Arab | 1.37 | 0.54 | 8% | 62% | 28 | CR |
| C05-T-03 | Cross breed | 0.10 | 0.10 | −23% | −36% | 28 | CR |
| C05-T-04 | Cattle dog x | 0.30 | 0.12 | 39% | 71% | 28 | CR |
| C05-T-05 | Shi Tzu | 0.29 | 0.11 | −12% | 61% | 28 | CR |
| C05-T-07 | Staffordshire Terrier | 0.08 | 0.10 | 0% | 13% | 28 | CR |
| C05-T-08 | Bull Mastiff x | 0.24 | 0.10 | 16% | 59% | 28 | CR |
| C05-T-09 | German Shepherd X | 1.32 | 0.53 | −8% | 13% | 28 | PD |
| C05-T-12 | Staffordshire Terrier | 0.72 | 0.29 | 0% | 23% | 28 | CR |
| C05-T-13 | | 0.23 | 0.10 | 28% | 35% | 28 | CR |
| C05-T-15 | | 0.14 | 0.10 | 12% | 45% | 28 | CR |
| C05-T-14 | Boxer | 0.20 | 0.10 | 20% | 27% | 28 | CR |
| C05-T-17 | Maltese Terrier | 2.97 | 1.20 | 6% | 31% | 28 | SD |
| C05-T-18 | Jack Russell Terrier | 0.81 | 0.32 | 34% | 76% | 21, 37 | CR |
| C05-T-19 | Jack Russell Terrier | 0.31 | 0.13 | 20% | 59% | 28 | CR |
| C05-V-01 | Boxer | 0.40 | 0.20 | — | — | 21, 37 | PR |
| C05-V-02 | Staffordshire Terrier | 4.97 | 2.00 | −26% | −118% | 21 | PD |
| C05-N-01 | Crossbred Terrier | 3.90 | 1.56 | −20% | 14% | 28 | CR |
| | | | Mean | 5% | 27% | | |
| | | | Median | 6% | 31% | | |

ID = identification;
CR = complete response;
PR = partial response;
SD = stable disease;
PD = progressive disease

TABLE 29
Non-CR RECIST results

| | | | | Tumor response | | | |
|---|---|---|---|---|---|---|---|
| | | | | following 24 h | | | |
| | | | | prednisolone | | | |
| | | | | administration | | | |
| | | | | (screening to | | | |
| | | | | immediate pre- | | | |
| | | Calculated | | treatment) | | Efficacy assessment | |
| | | tumor | Dose | | Tumor | Day of | |
| | | volume | Volume | RECIST | vol (cm³) | RECIST | RECIST |
| Case ID | Breed | (cm³) Day 0 | (mL) | reduction | reduction | assessment(s) | classification |
| C05-T-09 | German Shepherd X | 1.32 | 0.53 | −8% | 13% | 28 | PD |
| C05-T-17 | Maltese Terrier | 2.97 | 1.20 | 6% | 31% | 28 | SD |
| C05-V-01 | Boxer | 0.40 | 0.20 | — | — | 21, 37 | PR |
| C05-V-02 | Staffordshire Terrier | 4.97 | 2.00 | −26% | −118% | 21 | PD |

Dog C05-T-09 had a raised, hairless cutaneous MCT located on the mid-left abdomen medial to the mammary fold. Pre-treatment evaluation included a veterinary description of ‘infected’ with a ‘pink/red’ colour. It was an anxious rescue dog that had been with the owner for only a short time and was referred from a veterinary practice at least 4 h road travel time from the attending veterinarian. The tumor volume was 1.3 cm³ on pre-treatment evaluation and had increased by 8% RECIST measure and 13% tumor volume from screening (Day −2) and following 24 h neoadjuvant prednisolone immediately prior to drug treatment. The animal was examined by the attending veterinarian until the Day 7 visit. After this time, the owner declined to return to study and had monitoring visits conducted at another clinic (WSVC). Beyond Day 7 post treatment, the tumor continued to grow. In the absence of attending veterinarian care, tumor retreatment with the drug was not possible. On Day 33, the mass was surgically excised. Histopathology indicated a Patnaik Grade III tumor at that time.

Dog C05-T-17 had a raised subcutaneous MCT located above the stifle on the caudolateral surface of the left hind leg. The tumor volume was 3.0 cm on pre-treatment evaluation and had decreased by 6% RECIST measure and 31% tumor volume from screening (Day −1) and following 24 h neoadjuvant prednisolone immediately prior to drug treatment. Residual tissue growth was first noted by the attending veterinarian on Day 28 post treatment. The dog was retreated with a CR RECIST result reported on Day 42 post repeat treatment (data does not form part of this study).

Dog C05-V-01 had a raised, hairless cutaneous MCT located on the left ventral neck. The tumor volume was 0.4 cm³ on pre-treatment evaluation. Tumor growth response to 24 h neoadjuvant prednisolone was unavailable as a screening tumor evaluation was not conducted. The dog was a referral case examined first by the attending veterinarian on the day of treatment. The tumor experienced a 60% RECIST reduction by Day 37. However, the owners moved interstate and were unable to attend further follow up appointments. On Day 57, the residual mass (70% RECIST reduction; data not shown) was surgically removed. Histopathology indicated excision with complete margins, a central haemorrhagic MCT of Patnaik Grade II.

Dog C05-V-02 was a raised, hairless and ulcerative cutaneous MCT located on the right side vulva (mucocutaneous zone). Due to the location and size of tumor, surgery was not considered possible. The tumor volume was 5.0 cm³ on pre-treatment evaluation and had increased by 26% RECIST measure and 118% tumor volume from screening (Day −8) and following 24 h neoadjuvant prednisolone immediately prior to drug treatment. After initial tumor reduction and dissipation following Compound 1 treatment, residual tumor mass was evident by Day 21 it was decided to inject neoadjuvant triamcinolone (Day 25) and surgically resect tumor and inject Compound 1 into surgical margins (Day 35) (data does not form part of this study). Histopathology indicated a Patnaik Grade III MCT.

With the exception of swelling and pain, local tissue pathology as a result of treatment (i.e. tumor necrosis) was not considered an adverse event (AE). AEs reported such as transitory post treatment lethargy, listlessness and apathy are generally considered to be the result of the powerful local inflammatory response and systemic activation of the dog's immune response during the treatment response phase. In this study, 72 AEs were identified in 14 event types (see Table 28). The majority of identified AEs (59 out of 72; 82%) were represented by the ‘mild’ VCOG-CTCAE classification Grade 1 with no intervention required. With the exception of dogs C05-T-04 and C05-T-17 (described previously), a level of minimal medical intervention was required in only 3 cases. Dog C05-T-05 was medicated with metronidazole for transient diarrhea reported on Day 14 post-treatment; dog C05-T-07 was medicated with an antibiotic course (amoxicillin/clavulanic acid) for skin pustulation on the ventral abdomen (distant to drug injection site); and dog C05-T-09 was prescribed a prophylactic antibiotic course (cephalexin) on Day 21 on observation by the referral practice of ‘inflamed and angry’ lesion edges and having an impression smear of wound surface with ‘neutrophils and round cells (without granules)’. These findings for dog C05-T-09 of neutrophil presence is perhaps not unexpected due to the mechanism of action of the drug. The dog was reported having normal demeanour at this time. The incidence rates of each reported AE and associated VCOG-CTCAE grade classification are summarised in Table 30. With the exception of the limb swelling in dog C05-T-17 (degranulation reaction), all reported AEs were transient in nature—resolved by 24-48 h post treatment.

TABLE 30
Incidence rates and VCOG-CTCAE classification
of adverse events (AEs)

| | | | VCOG- | |
|---|---|---|---|---|
| | | | CTCAE | |
| | All dogs | | Grade | |
| | No. of dogs | | classification | |
| | experiencing | Incidence | (no. of | |
| Event type | this event | (%) | animals) | |
| Local swelling at treatment | 18 | 100.0 | 1 (17) | 2 (1) |
| site* | | | | |
| Pain associated with treatment | 13 | 72.2 | 1 (12) | 2 (1) |
| site* | | | | |
| Bruising at treatment site* | 14 | 77.8 | 1 (13) | 2 (1) |
| Lethargy | 6 | 33.3 | 1 (5) | 2 (1) |
| Listless | 2 | 11.1 | 1 (2) | — |
| Apathy | 2 | 11.1 | 1 (2) | — |
| Inappetance | 7 | 38.9 | 1 (6) | 2 (1) |
| Transient Diarrhoea | 2 | 11.1 | 1 (1) | 2 (1) |
| Vomiting | 3 | 16.7 | 1 (2) | 2 (1) |
| Cranial abdominal pain | 1 | 5.6 | — | 2 (1) |
| Injection site lesion | 1 | 5.6 | — | 2 (1) |
| antimicrobial prophylaxis | | | | |
| Skin pustulation | 1 | 5.6 | — | 2 (1) |
| Ringworm | 1 | 5.6 | 1 (1) | — |
| Polydipsia | 1 | 5.6 | 1 (1) | — |

*Not an unexpected outcome due to the mechanism of action of the drug

This study investigated the efficacy and safety of Compound 1 injection for MCT treatment in the presence of oral prednisolone under defined clinical situations. Of the eighteen (18) cases enrolled in the study, fourteen (14) (78%) achieved a complete response (CR), 1 achieved partial response (PR), 1 achieved stable disease (SD), and two (2) experienced progressive disease. Prednisolone is expected to have only physiological and anti-inflammatory effects at the dose given (1 mg/kg/day) (Mason, 2012). As the level of efficacy achieved is similar to previous studies, results suggest that oral prednisolone provided for 24 h pre-treatment and 7 days post-treatment at the above dose level exerts limited or no suppressive effects on drug action.

### Example 6: Treatment of Soft Tissue Sarcoma with Compound 1

This study demonstrate the efficacy of Compound 1 in the treatment of soft tissue sarcoma in dogs (client owned, domestic dogs).

### Eligibility

Dogs were selected for the study if they met the criteria in Table 1 (Example 1) but where the dog has at least one STS. Study dogs were managed with due regard for their welfare. All animals were client-owned, treated by Australian registered veterinarians, and cases managed under their direct supervision. An enrolled dog could be removed from the study prior to study completion under the following circumstances:
1. If at any time during the study, in the opinion of the attending veterinarian, the dog's welfare is at risk due to non-response of the tumor to Compound 1 treatment or due to a serious adverse event, the veterinarian could elect to withdraw the dog from the study. Following withdrawal, the dog's attending veterinarian could then administer supportive treatment and veterinary care that they deem is appropriate and in the best interest of the animal.
2. The owner elected to voluntarily withdraw the dog from the study.

Any dog removed from the study received appropriate veterinary care.

### Study Procedure

1. Only one tumor per animal is treated.
2. The dog received prophylactic medications as deemed appropriate by the supervising veterinarian prior to dosing.
3. A clinical examination was performed prior to treatment.
4. The total calculated volume of the selected STS was confirmed as ≤12.5 cm3 (determined as ½×length (cm)×width (cm)×depth (cm)).
5. The calculated dose of drug for the dog was confirmed as ≤0.25 mg Compound 1 per kg at the Day 0 visit (maximum dose of 5 mg Compound 1).
6. The drug was provided to veterinarians as a 1 mg/mL Compound 1 formulation in ready to use form.
7. The required volume of the dose concentration of Compound 1 was drawn into an appropriate luer lock syringe with a 23-27 gauge needle.
8. The drug was delivered throughout the tumor in a fractionated manner that ensures even distribution.
9. Treated tumors were observed regularly and observations recorded at days 1, 7 (+2), 14 (+2) and 28 (+2) days post-treatment.
10. Tumors may be cleaned with clean water or saline as necessary until healed. No additional treatment was provided without attending veterinarian approval.
11. Retreatment was offered (if required) after 28 days.

Supporting prophylactic therapies are shown in Table 31.

TABLE 31
Prophylactic and Supportive Medications

| | | | | Dog ID | | | | | | | | | | | |
|---|---|---|---|---|---|---|---|---|---|---|---|---|---|---|---|
| | | | | | | C04-T- | | C04-T- | | | | | | | C04-T- |
| Prophylactic and Supportive Medications | | | | C04- | C04- | 02 | C04- | 03 | C04- | C04- | C04- | C04- | C04- | C04- | 09 |
| | Active ingredient | Tradename | Delivery | T-01 | T-02 | (repeat) | T-03 | (repeat) | T-04 | T-05 | T-06 | T-07 | T-08 | T-09 | (repeat) |
| Pre- | Sotalol | APO- | PO | X | | | | | | | | | | | |
| Tx | | SOTALOL | | | | | | | | | | | | | |
| | Meloxicam | METACAM | SC | X | X | X | X | X | X | X | X | X | X | X | X |
| | Amoxicillin + | AMOXYCLAV | PO | | | | X | | X | | | | | | |
| | Clavulanic acid | | | | | | | | | | | | | | |
| | Prednisolone | MACROLONE | PO | | | | | | X | | | | | | |
| Post- | Sotalol | APO- | PO | X | | | | | | | | | | | |
| Tx | | SOTALOL | | | | | | | | | | | | | |
| | Prednisolone | MACROLONE | PO | | | | | | X | | | | | | |
| | Chlorpheniramine | IRAMINE | PO | | | | | | | X | | | | | |
| | Famotidine | FAMOTIDINE | PO | | | | | | | X | | | | | |
| | Tramadol | TRAMADOL | PO | X | X | X | X | X | | X | X | X | X | X | X |
| | Meloxicam | METACAM | PO | X | | | | | | X | X | X | X | X | X |
| | Carprofen | RIMADYL | PO | | X | X | X | X | | | | | | | |
| | IV saline | | IV | | | | | | | X | | | | | |
| | Amoxicillin + | NOROCLAV | SC | | | | | | | | | | X | | |
| | Clavulanic acid | | | | | | | | | | | | | | |
| | Amoxicillin + | AMOXYCLAV | PO | | | | | | | | | | X | | |
| | Clavulanic acid | | | | | | | | | | | | | | |
| | Neomycin + | APEX | TP | | | | | | | | | | | X | |
| | Hydrocortisone + | LOTION | | | | | | | | | | | | | |
| | Lignocaine | | | | | | | | | | | | | | |
| | Chlorhexidine | PYOHEX | TP | | | | | | | | | | | | X |
| | | SHAMPOO | | | | | | | | | | | | | |
| | Dexamethasone | DEXAFORT | IM | | | | | | | | | | | | X |
| | Meloxicam | METACAM | SC | | | | | | | X | | | | | |

ID = identification;
Tx = treatment;
IV = intravenous;
IT = intratumoural;
PO = per os (by mouth);
PR = per rectum;
SC = subcutaneous;
TP = topical

### Results

The results are shown in Tables 32 and 33.

TABLE 32

| | Calc. | Compound 1 | | |
|---|---|---|---|---|
| | tumor vol | conc. | | RECIST |
| NO./Breed | (cm³) Day 1 | (mg/mL) | Dose volume | classification |
| 1/Boxer | 0.71 | 1.0 | 0.3 | CR |
| 2/Cattle Dog | 10.3 (0.72) | 1.0 | 4.00 (0.3) | PR (CR) |
| 3/Staffordshire | 16.7 (1.26) | 1.0 | 4.5 (0.50) | PR (CR) |
| Terrier X | | | | |
| 4/Bulldog X | 2.03 | 1.0 | 81 | CR |

TABLE 33

| | Month of Case follow up after last treatment (ongoing) | | | | | | | | | | | |
|---|---|---|---|---|---|---|---|---|---|---|---|---|
| No | 1 | 2 | 3 | 4 | 5 | 6 | 7 | 8 | 9 | 10 | 11 | 12 |
| 1 | NT | | NT | | | NT | | | NT | | | NT |
| 2* | | NT | | | NT | | | NT | | | NT | |
| 3* | | NT | | | NT | | | NT | | | | |
| 4 | | | NT | | | | | | | | | |

NT = No Tumor
*repeat treatment occurred, results are after repeat treatment.

### Example 7: Treatment of MCT

The study of Example 1 was repeated. The drug used was Compound 1 formulated in 40% propylene glycol and 30 MM acetate buffer at pH 4.2.

While tumor evaluation and measurement data for 11 cases are presented, this actually represents only 10 dogs, one of which had two tumors treated consecutively (identified as cases SI-01 and SI-02). PK sampling was performed only once for this dog (SI-01).

Demographics of the enrolled cases by case ID are summarized in Table 34. Of the ten enrolled dogs (SI-02 data were not included in demographics totals or means), there were four males (one intact) and six spayed females. The mean age was 8.9 years (range 6.0-14.0 years) and the mean body weight on day 0 was 25.9 kg (range 6.9-44.5 kg). The enrolled study population involved seven purebred dogs and three mixed breed dogs. The Labrador Retriever (purebred and mix) was the most common breed type represented.

A total of six cutaneous and five subcutaneous tumors were studied in the enrolled dogs. MCT location was variable; however, the majority of tumors (8/11, 72.7%) were located on or near the limb (e.g., foot, leg, hip, thigh, flank). MCTs for the three other cases were located at the pinnal base, the chest, and the perianal region.

TABLE 34
Demographics by Case ID

| | | | | | Day 0 | | |
|---|---|---|---|---|---|---|---|
| | | Age at | | | body | | |
| Case | | screening | | Reproductive | weight | Tumor | Tumor |
| ID | Breed | (yr) | Gender | status | (kg) | location | type |
| SI-01 | Labrador | 7.0 | F | Spayed | 27.1 | Dorsal, right | Cutaneous |
| | Retriever | | | | | front foot | |
| | mix | | | | | | |
| SI-02 | Labrador | 7.1 | F | Spayed | 26.6 | Dorsal, mid | Cutaneous |
| | Retriever | | | | | left foreleg | |
| | mix | | | | | | |
| SI-03 | Doberman | 8.5 | M | Neutered | 33.2 | Left perianal | Cutaneous |
| | Pinscher | | | | | region | |
| SI-04 | Labrador | 8.9 | M | Intact | 44.5 | Right medial | SQ |
| | Retriever | | | | | flank | |
| SI-05 | English | 7.9 | F | Spayed | 23.3 | Medial | Cutaneous |
| | Bulldog | | | | | aspect of left | |
| | | | | | | pinna base | |
| SI-06 | Vizsla | 8.4 | F | Spayed | 21.3 | Ventral | SQ |
| | | | | | | caudal chest | |
| SI-07 | Golden | 14.0 | F | Spayed | 32.8 | Left caudal | SQ |
| | Retriever | | | | | distal thigh | |
| SI-08 | Boxer | 6.2 | F | Spayed | 23.5 | Dorsal, right | SQ |
| | | | | | | hip | |
| SI-09 | Jack | 12.0 | M | Neutered | 19.5 | Right | SQ |
| | Russell | | | | | lateral thigh | |
| | Terrier | | | | | | |
| | mix | | | | | | |
| SI-10 | Silky | 6.0 | M | Neutered | 6.9 | Dorsal, right | Cutaneous |
| | Terrier | | | | | hip | |
| SI-11 | Labrador | 10.4 | F | Spayed | 27.3 | Left leg, | Cutaneous |
| | Retriever | | | | | cranial of | |
| | mix | | | | | tibia | |

SQ = subcutaneous

Of the ten dogs treated, two cases (SI-01/02 and SI-08) had a history of previous MCT and/or multiple MCTs at enrollment. The majority of the other dogs were enrolled with various types of benign and malignant tumors.

There was slight leakage of IVP upon injection for three cases (SI-01 with <1% loss; SI-02 and SI-04 with <10% loss), and moderate leakage for two cases (SI-05 and SI-10 with 20% loss). Despite the leakage, all five cases were determined by the investigator to be a complete response on or before the day 14 visit.

Injection site abnormalities were reported within the immediate post-injection period for six cases. Three cases experienced pain and three cases exhibited restlessness, all of which resolved by day 1. An analgesic was prescribed to start on day 0 for one of the six cases (SI-11). The other five cases did not receive medical intervention during the immediate post-injection period. The VCOG CTCAE classifications of these injection site abnormalities for magnitude and relationship to IVP as determined by the investigator are listed in Table 35.

TABLE 35
IVP dosing information

| | Calc. | | | | Est. IVP | | | |
|---|---|---|---|---|---|---|---|---|
| | tumor | Compound 1 dose | | | leakage | | | |
| | volume | mg/kg | | mg/cm³ | from | | VCOG CTCAE | |
| Case | on day 0 | body | total | of tumor | tumor | Injection site | | Relationship |
| ID | (cm³)^{a} | weight^{b} | mg^{c} | volume^{d} | (mL) | abnormality^{e} | Magnitude | to IVP^{g} |
| SI- | 2.6 | 0.05 | 1.3 | 0.50 | 0.01 | Injection | Grade 1^{f} | Probable |
| 01 | | | | | | site pain | | |
| SI- | 5.1 | 0.1 | 2.6 | 0.51 | 0.2 | | — | — |
| 02 | | | | | | | | |
| SI- | 1.9 | 0.03 | 1.0 | 0.53 | 0 | Restlessness | Grade 1 | Definite |
| 03 | | | | | | (upon | | |
| | | | | | | injection) | | |
| SI- | 5.6 | 0.06 | 2.8 | 0.50 | 0.2 | | — | — |
| 04 | | | | | | | | |
| SI- | 0.1 | 0.002 | 0.05 | 0.50 | 0.01 | | — | — |
| 05 | | | | | | | | |
| SI- | 4.0 | 0.09 | 2.0 | 0.50 | 0 | Restlessness | Grade 1 | Definite |
| 06 | | | | | | (upon | | |
| | | | | | | injection) | | |
| SI- | 3.1 | 0.05 | 1.6 | 0.52 | 0 | | — | — |
| 07 | | | | | | | | |
| SI- | 3.3 | 0.07 | 1.7 | 0.52 | 0 | | — | — |
| 08 | | | | | | | | |
| SI- | 3.2 | 0.08 | 1.6 | 0.50 | 0 | Restlessness | Grade 1 | Definite |
| 09 | | | | | | (upon | | |
| | | | | | | injection) | | |
| SI- | 2.0 | 0.14 | 1.0 | 0.50 | 0.2 | Injection | Grade 1 | Possible |
| 10 | | | | | | site pain | | |
| SI- | 6.8 | 0.12 | 3.4 | 0.50 | 0 | Injection | Grade 1 | Definite |
| 11 | | | | | | site pain | | |

^{a}Tumor volume calculated as 0.5 × L × W × D
^{b}Based on day 0 body weight (kg).
^{c}IVP was 1 mg EBC-46 API per mL, therefore dose of IVP in mg equals dose in mL.
^{d}Based on day 0 tumor volume (cm³)
^{e}Recorded as VeDDRA LLT
^{f}Grade 1: Mild; asymptomatic or mild symptoms; clinical signs or diagnostic observations only; intervention not indicated.
^{g}Unrelated: Clearly not related to IVP; Unlikely: Doubtfully related to IVP; Possible: May be related to IVP; Probably: Likely related to IVP; Definite: Clearly related to IVP.

The tumor volumes at the screening visit ranged from 0.3 to 9.7 cm³. Between screening and day 0, tumors for most cases decreased in volume after the FNA procedure and antihistamine administration, while volumes for three cases (SI-03, SI-10, and SI-11) increased. The tumor volumes on day 0 ranged from 0.1 to 6.8 cm³. Volumes for all tumors decreased in size after drug treatment with the exception of SI-03, a case that exited the study after the day 28 visit due to lack of response to the drug treatment.

The number and cumulative percent of tumors with complete response and with healing by scheduled time point are presented in Table 33. Ten (90.9%) of 11 tumors exhibited a complete response (CR) during the study, defined as a disappearance of the target lesion. The MCT for SI-03 continued to enlarge despite drug treatment so the dog was withdrawn from the study after completing the day 28 visit for the owner to pursue alternate treatments. Of the other ten tumors, nine were considered CR by day 7 (+2) and one was CR by day 14 (+2). The investigator also reported that all of these tumors remained a CR on day 28 (+4). At the day 28 examination, one case (SI-10) exhibited a ridge of tissue near the original tumor site. The investigator conducted an FNA, which revealed MCT, which the investigator believed to be a new tumor rather than a recurrence.

Healing was more variable for the cases, with one healed at the day 14 visit and over half the tumor sites healed by the day 28 visit. The two cases with the largest tumor volumes at day 0 (SI-04 5.6 cm³, and SI-11 6.8 cm³) required healing time beyond the day 28 visit, i.e., an additional four weeks for SI-04 and an additional 19.7 weeks for SI-11. For SI-11, it was noted the dog was licking the tumor site and the investigator felt it may have prolonged the healing time. The investigator believed the wound required intervention on this case that included debridement once on study day 56 followed by periodic bandage replacement.

TABLE 36
Number and cumulative percent of tumors with complete response and
healing by scheduled time point

| | | | | Healing | | |
|---|---|---|---|---|---|---|
| | | | | Number | | |
| | Complete response | | | of tumor | | |
| | Number | | | sites | | |
| | of tumors | | | healed on | | |
| Study | on given | | | given | | |
| day | study day | Cumulative % | Case ID | study day | Cumulative % | Case ID |
| 7 (+2) | 9 | 82 | SI-02, SI- | | | |
| | | | 04, SI-05, | | | |
| | | | SI-06, SI- | | | |
| | | | 07, SI-08, | | | |
| | | | SI-09, SI- | | | |
| | | | 10, SI-11 | | | |
| 14 (+2) | 1 | 91% | SI-01 | 1 | 9% | SI-05 |
| 28 (+4) | | | | 5 | 55% | SI-01, SI- |
| | | | | | | 02, SI-06, |
| | | | | | | SI-07, SI- |
| | | | | | | 08 |
| 33 | | | | 1 | 64% | SI-10 |
| 42 | | | | 1 | 73% | SI-09 |
| 56 | | | | 1 | 82% | SI-04 |
| 166 | | | | 1 | 91% | SI-11 |

Note:
Percentages are based on 11 treated tumors in 10 dogs, rounded up to the nearest whole number. Due to tumor enlargement, SI-03 was removed from the study after the day 28 visit prior to CR to initiate chemotherapy.

### Example 7: Topical Treatment of Tumors

Compound 1 was formulated as a gel composition in isopropanol, water and Carbomer 940 at 5 mg Compound 1 per mL. The gel formulation was applied directly to the surface of the tumor lesion.

### REFERENCES

It is to be understood that, if any prior art publication is referred to herein, such reference does not constitute an admission that the publication forms a part of the common general knowledge in the art, in Australia or any other country.
Blackwood, L., Murphy, S., Buracco, P., De Vos, J. P., De Fornel-Thibaud, P., Hirschberger, J., Kessler, M., Pastor, J., Ponce, F., Savary-Bataille, K. and Argyle, D. J. (2012) European consensus document on mast cell tumours in dogs and cats. Vet Comp Oncol: doi:10.1111/j.1476-5829.2012.00341.x.
Celikoglu, F., Celikoglu, S. I., and Goldberg, E. P. (2008) Techniques for intratumoural chemotherapy of lung cancer by bronchoscopic drug delivery. Cancer Therapy 6:545-552.
Eisenhauer, E. A., Therase, P., Bogaerts, J., Schwartz, L. H., Sargent, D., Ford, R., Dancey, J., Arbuck, S., Gwyther, S., Mooney, M., Rubinstein, L., Shankar, L., Dodd, L., Kaplan, R., Lacombe, D., and Verweij, J. (2009) New response evaluation criteria in solid tumours: revised RECIST guideline (version 1.1), Eur J Cancer 45(2): 228-247.
Faustino-Rocha, A., Oliveira, P. A., Pinho-Oliveira, J. et al.: Estimation of rat mammary tumor volume using caliper and ultrasonography measurements, Lab Dogs (NY) 42(6):217-224, 2013.
Fox, F. E., Rosentahl, R. C., Twedt, D. C., Dubielzig, R. R., MacEwen, E. G. and Grauer, G. F. (1990) Plasma histamine and gastrin concentrations in 17 dogs with mast cell tumors. J Vet Intern Med 4(5):242-246.
Ishiguro, T., Kadosawa, T., Takagi, S., Kim, G., Ohsaki, T. and Bosnakovski, D. (2003) Relationship of disease progression and plasma histamine concentrations in 11 dogs with mast cell tumors. J Vet Intern Med 17(2): 194-198. doi: 10.11111/j.1939-1676.2003.tb02433.x.
Koplitz, S. L., Scott, M. A., Cohn, L. A. (2001) Effects of platelet clumping on platelet concentrations measured by use of impedance or buffy coat analysis in dogs. J. Am. Vet. Med. Assoc. 219(11): 1552-1556.
Mason, D (2012) Prednisone therapy in dogs and cats (Veterinary Emergency Clinic Internal Medicine News). Available from: vectoronto.com/content/uploads/2012/12/IntemalMedicineNews4.pdf [16 Dec. 2015].
Monga, S. P., Wadleigh, R., Sharma, A., Adib, H., Strader, D., Singh, G., Harmon, J. W., Berlin, M., Monga, D. K., and Mishra, L. (2000) Intratumoural therapy of cisplatin/epinephrine injectable gel for palliation in patients with obstructive esophageal cancer. Am J Clin Oncol (CCT) 23: 386-392.
Pimental, T. A., Sampaio, A. L. F., D'Acquisto, F., Perretti, M. and Oliani, S. M. (2011) Blood, lymphatics, immune system, stem cells. An essential role for mast cell as modulators ofneutrophils influx in collagen-induced arthritis in the mouse. Lab Invest. 91: 33-42, 2011.
Thompson et al.: Canine subcutaneous mast cell tumor: characterization and prognostic indices. Vet. Pathol. 48(1): 156-168, 2011.
Veterinary Cooperative Oncology Group—common terminology criteria for adverse events (VCOG-CTCAE) following chemotherapy or biological antineoplastic therapy in dogs and cats v1.1, Vet Comp Oncol. July 2011. Available from: <http://www.vetcancersociety.org/members/files/2012/01/CTCAE-v1-1.pdf>[18 Mar. 2015].

1. A method of treating a mast cell tumor or a soft tissue sarcoma in a subject comprising administering to the subject a compound of formula (I): 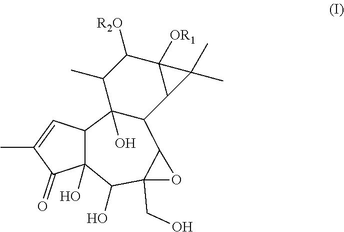
wherein R₁ and R₂ are independently selected from —C₁₋₂₀alkyl, —C₂₋₂₀alkenyl, —C₂₋₂₀alkynyl, —C(O)C₁₋₂₀alkyl, —C(O)C₂₋₂₀alkenyl and —C(O)C₂₋₂₀alkynyl;
wherein each alkyl, alkenyl and alkynyl may be optionally substituted with one or more groups selected from —OH, —SH, —OC₁₋₄alkyl, —SC₁₋₄alkyl, -halo, —CN, —CF₃, —CHF₂, —CH₂F, —OCF₃, —OCHF₂, —OCH₂F, —SCF₃, —SCHF₂, —SCH₂F, —CO₂H, —CO₂C₁₋₄alkyl, —NH₂, —NH(C₁₋₄alkyl), —N(C₁₋₄alkyl)₂ and —NO₂;
or a pharmaceutically acceptable salt thereof; in combination with at least one other pharmaceutically active agent.

2. The method according to claim 1 wherein R₁ and R₂ are independently selected from —C₁₋₁₀alkyl, —C₂₋₁₀alkenyl, —C₂₋₁₀alkynyl, —C(O)C₁₋₁₀alkyl, —C(O)C₂₋₁₀alkenyl and —C(O)C₂₋₁₀alkynyl.

3. The method according to claim 2 wherein R₁ and R₂ are independently selected from —C(O)C₁₋₁₀alkyl, —C(O)C₂₋₁₀alkenyl and —C(O)C₂₋₁₀alkynyl.

4. The method according to claim 3 wherein R₁ and R₂ are independently selected from —C(O)C₁₋₁₀alkyl and —C(O)C₂₋₁₀alkenyl.

5. The method according to claim 1 wherein R₁ is selected from —C(O)C₁₋₁₀alkyl.

6. The method according to claim 1 wherein R₂ is selected from —C(O)C₁₋₆alkyl and —C(O)C₂₋₆alkenyl.

7. The method according to claim 1 wherein compound of formula (I) is selected from:
12-tigloyl-13-(2-methylbutanoyl)-6,7-epoxy-4,5,9,12,13,20-hexahydroxy-1-tigliaen-3-one,
12,13-di-(2-methylbutanoyl)-6,7-epoxy-4,5,9,12,13,20-hexahydroxy-1-tigliaen-3-one,
12-hexanoyl-13-(2-methylbutanoyl)-6,7-epoxy-4,5,9,12,13,20-hexahydroxy-1-tigliaen-3-one, and
12,13-di-hexanoyl-6,7-epoxy-4,5,9,12,13,20-hexahydroxy-1-tigliaen-3-one; or a pharmaceutically acceptable salt thereof.

8. The method according to claim 1 wherein the tumor is a mast cell tumor.

9. The method according to claim 1 wherein the tumor is a soft tissue sarcoma.

10. The method according to claim 9 wherein the soft tissue sarcoma is selected from the group consisting of fibrosarcoma, malignant fibrous hystiocytoma, dermatofibrosarcoma, liposarcoma, myxosarcoma, rhabdomyosarcoma, malignant mesenchymoma, leiomyosarcoma, hemangiosarcoma, Karposi's sarcoma, lymphangiosarcoma, undifferentiated sarcoma, synovial sarcoma, peripheral nerve sheath tumor, neurofibrosarcoma, pleomorphic sarcoma, extraskeletal chondrosarcoma, extraskeletal osteosarcoma, embryonyl sarcoma, infantile haemangiopericytoma (perivascular wall tumor), malignant schwannoma, neurogenic sarcoma, alveolar soft part sarcoma, extraskeletal myxoid chondrosarcoma or extraskeletal mesenchymal sarcoma.

11. The method according to claim 1 wherein the subject is a human, companion animal or a farmed animal.

12. (canceled)

13. The method according to claim 11 wherein the companion animal or farmed animal is selected from a dog, cat, horse, cow and pig.

14. The method according to claim 13 wherein the subject is a dog.

15. The method according to claim 1 wherein the administration of the compound of formula (I) is localised administration.

16. The method according to claim 15 wherein the localised administration is by intratumoral injection or by topical application.

17. (canceled)

18. The method claim 1 wherein the at least one further pharmaceutically active agent is selected from an antihistamine; an anti-inflammatory agent or a compound that has gastric protection activity or an antihistamine and an anti-inflammatory agent or an antihistamine, an anti-inflammatory agent and a compound that has gastric protection activity.

19. (canceled)

20. A method of treating a mast cell tumour in a subject comprising administering a compound of formula (I) in combination with at least one anti-inflammatory compound and at least one antihistamine; wherein the compound of formula (I) is 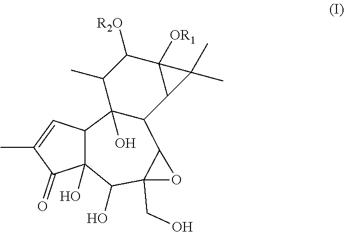
wherein R₁ and R₂ are independently selected from —C₁₋₂₀ alkyl, —C₂₋₂₀alkenyl, —C₂₋₂₀alkynyl, —C(O)C₁₋₂₀alkyl, —C(O)C₂₋₂₀alkenyl and —C(O)C₂₋₂₀alkynyl:
wherein each alkyl, alkenyl and alkynyl may be optionally substituted with one or more groups selected from —OH, —SH, —OC₁₋₄alkyl, —SC₁₋₄alkyl, -halo, —CN, —CF₃, —CF₂, —CH₂F, —OCF₃, —OCHF₂, —OCH₂F, —SCF₃—SCHF₂, —SCH₂F, —CO₂H, —CO₂C₁₋₄alkyl, —NH₂, —NH(C₁₋₄alkyl), —N(C₁₋₄alkyl)₂ and —NO₂;
or a pharmaceutically acceptable salt thereof.

21. A method according to claim 20 further comprising administering a compound that has gastric protection activity.

22. (canceled)

23. A kit comprising a compound of formula (I) as defined in claim 1 or a pharmaceutically acceptable salt thereof and at least one second pharmaceutically active agent.

24. The kit according to claim 23 wherein the at least one second pharmaceutically active agent is an antihistamine or a corticosteroid or an antihistamine and a corticosteroid.
